(19) United States
(12) Patent Application Publication
(10) Pub. No.: US 2024/0428920 A1
(43) Pub. Date: Dec. 26, 2024

(54) OROFACIAL MYOFUNCTIONAL THERAPY METHODS AND SYSTEMS

(71) Applicant: Vivos Therapeutics, Inc., Highlands Ranch, CO (US)

(72) Inventors: Dean F. Raio, Dix Hills, NY (US); Giselle Raio, Dix Hills, NY (US)

(73) Assignee: Vivos Therapeutics, Inc., Highlands Ranch, CO (US)

(21) Appl. No.: 18/778,786

(22) Filed: Jul. 19, 2024

Related U.S. Application Data
(63) Continuation of application No. PCT/US2023/061237, filed on Jan. 25, 2023.
(60) Provisional application No. 63/302,870, filed on Jan. 25, 2022.

(51) Int. Cl. G16H 20/30; A61F 5/00; A63B 23/03

(52) U.S. Cl. CPC G16H 20/30; A61F 5/00; A63B 23/032

(57) ABSTRACT

Described herein are methods and systems for using orofacial myofunctional therapy to non-surgically treat orofacial muscle disorders (OMDs) in a patient. The methods and systems combine orofacial myofunctional therapy exercises with an oral appliance. The methods and systems may include a three-phase orofacial myofunctional therapy program that can vary in length depending on patient diagnosis and evaluation by a healthcare professional.

### RELATED APPLICATIONS

This application is a continuation of, and claims priority to PCT Application No. PCT/US2023/061237 filed Jan. 25, 2023, entitled “OROFACIAL MYOFUNCTIONAL THERAPY METHODS AND SYSTEMS”. The '237 application claims priority to, and benefit of, U.S. Provisional Application Ser. No. 63/302,870, filed on Jan. 25, 2022, entitled “OROFACIAL MYOFUNCTIONAL THERAPY METHODS AND SYSTEMS.” Both applications are hereby incorporated by reference in their entirety for all purposes.

### FIELD OF DISCLOSURE

The present disclosure is directed to methods and systems for using orofacial myofunctional therapy to non-surgically treat orofacial muscle disorders (OMDs) in a patient. The methods and systems combine orofacial myofunctional therapy exercises with an oral appliance. The methods and systems may include a three-phase orofacial myofunctional therapy program that can vary in length depending on patient diagnosis and evaluation by a healthcare professional.

### BACKGROUND

OMDs are abnormal patterns of misuse of the muscles within the mouth and face that interfere with craniofacial development, growth, and/or the function of structures. OMDs are associated with a wide range of health problems, including mouth-breathing, temporomandibular joint dysfunction (TMD), upper airway difficulties, and sleep disordered breathing. Sleep disordered breathing may include snoring, upper airway resistance syndrome, and obstructive sleep apnea (OSA). Current treatment methods for OMDs include medical and surgical treatments. However, there is a critical need for a non-surgical treatment method to treat OMDs, including orthodontic, dentofacial, and/or craniofacial variations.

Oral appliances have proven successful in non-surgically treating OMDs, including orthodontic, dentofacial, and/or craniofacial variations, such as by utilizing cyclic forces to induce sutural osteogenesis via stem cell response to increase the size of, and change the shape of, the upper airway. However, there remains a need to stabilize foundational corrections as brought forth by oral appliances, and help patients learn how to use the foundational corrections, such as increased space within the upper airway.

Orofacial myofunctional therapy includes the completion of exercises by a patient that are specific to the orthodontic, dentofacial, and craniofacial variations of the patient. The exercises are designed to neurologically re-educate, re-pattern, and/or strengthen muscles that aid in the formation and maintenance of a healthy orofacial region. The orofacial region is the focal point of the upper airway and the area where obstructive sleep apnea and sleep disordered breathing occurs. This therapy results in a patient that will be a habitual nasal breather.

The present disclosure describes methods and systems for combining orofacial myofunctional therapy exercises with an oral appliance to non-surgically treat OMDs.

### BRIEF DESCRIPTION OF THE DISCLOSURE

In one aspect, provided herein is a method for treating an orofacial muscle disorder in a patient, the method including: (i) modifying a first component of an oral anatomy of the patient with an oral appliance; and (ii) exercising a second component of the oral anatomy of the patient with at least one orofacial myofunctional therapy exercise.

In another aspect, provided herein is a system for treating an orofacial muscle disorder in a patient, the system including: an oral appliance configured to modify a first component of an oral anatomy of the patient; instructions for performing at least one orofacial myofunctional therapy exercise to exercise a second component of the oral anatomy of the patient; and optionally at least one tool for performing the at least one orofacial myofunctional therapy exercise.

In yet another aspect, provided herein is a non-transitory computer-readable storage medium having computer-executable instructions embodied thereon for treating an orofacial muscle disorder in a patient, wherein the computing device is configured to be used in conjunction with an oral appliance configured to modify a first component of an oral anatomy of the patient and optionally at least one tool for performing at least one orofacial myofunctional therapy exercise, wherein when executed by a computing device including at least one processor in communication with a memory, the computer-readable instructions cause the computing device to: provide the patient with instructions for performing the at least one orofacial myofunctional therapy exercise to exercise a second component of the oral anatomy of the patient.

### BRIEF DESCRIPTION OF THE DRAWINGS

FIG. 1A depicts a resting tongue position.

FIG. 1B depicts a resting tongue position.

FIG. 1C depicts exercises targeting an improved resting tongue position in accordance with the present disclosure.

FIG. 1D depicts exercises targeting an improved resting tongue position in accordance with the present disclosure.

FIG. 1E depicts exercises targeting an improved resting tongue position in accordance with the present disclosure.

FIG. 1F depicts exercises targeting an improved resting tongue position in accordance with the present disclosure.

FIG. 2A depicts intrinsic and extrinsic muscles of a tongue.

FIG. 2B depicts intrinsic and extrinsic muscles of a tongue.

FIG. 2C depicts exercises targeting an increase in tongue strength in accordance with the present disclosure.

FIG. 2D depicts exercises targeting an increase in tongue strength in accordance with the present disclosure.

FIG. 2E depicts exercises targeting an increase in tongue strength in accordance with the present disclosure.

FIG. 2F depicts exercises targeting an increase in tongue strength in accordance with the present disclosure.

FIG. 2G depicts exercises targeting an increase in tongue strength in accordance with the present disclosure.

FIG. 2H depicts exercises targeting an increase in tongue strength in accordance with the present disclosure.

FIG. 2I depicts exercises targeting an increase in tongue strength in accordance with the present disclosure.

FIG. 2J depicts exercises targeting an increase in tongue strength in accordance with the present disclosure.

FIG. 2K depicts exercises targeting an increase in tongue strength in accordance with the present disclosure.

FIG. 2L depicts exercises targeting an increase in tongue strength in accordance with the present disclosure.

FIG. 2M depicts exercises targeting an increase in tongue strength in accordance with the present disclosure.

FIG. 2N depicts exercises targeting an increase in tongue strength in accordance with the present disclosure.

FIG. 2O depicts exercises targeting an increase in tongue strength in accordance with the present disclosure.

FIG. 2P depicts exercises targeting an increase in tongue strength in accordance with the present disclosure.

FIG. 3A depicts a genioglossus muscle of a tongue.

FIG. 3B depicts a genioglossus muscle of a tongue.

FIG. 3C depicts exercises targeting a genioglossus muscle of a tongue in accordance with the present disclosure.

FIG. 3D depicts exercises targeting a genioglossus muscle of a tongue in accordance with the present disclosure.

FIG. 3E depicts exercises targeting a genioglossus muscle of a tongue in accordance with the present disclosure.

FIG. 3F depicts exercises targeting a genioglossus muscle of a tongue in accordance with the present disclosure.

FIG. 3G depicts exercises targeting a genioglossus muscle of a tongue in accordance with the present disclosure.

FIG. 3H depicts exercises targeting a genioglossus muscle of a tongue in accordance with the present disclosure.

FIG. 3I depicts exercises targeting a genioglossus muscle of a tongue in accordance with the present disclosure.

FIG. 3J depicts exercises targeting a genioglossus muscle of a tongue in accordance with the present disclosure.

FIG. 3K depicts exercises targeting a genioglossus muscle of a tongue in accordance with the present disclosure.

FIG. 3L depicts exercises targeting a genioglossus muscle of a tongue in accordance with the present disclosure.

FIG. 4A depicts a styloglossus muscle of a tongue.

FIG. 4B depicts exercises targeting a styloglossus muscle of a tongue in accordance with the present disclosure.

FIG. 4C depicts exercises targeting a styloglossus muscle of a tongue in accordance with the present disclosure.

FIG. 4D depicts exercises targeting a styloglossus muscle of a tongue in accordance with the present disclosure.

FIG. 4E depicts exercises targeting a styloglossus muscle of a tongue in accordance with the present disclosure.

FIG. 4F depicts exercises targeting a styloglossus muscle of a tongue in accordance with the present disclosure.

FIG. 5A depicts a hyoglossus muscle of a tongue.

FIG. 5B depicts exercises targeting a hyoglossus muscle of a tongue in accordance with the present disclosure.

FIG. 5C depicts exercises targeting a hyoglossus muscle of a tongue in accordance with the present disclosure.

FIG. 5D depicts exercises targeting a hyoglossus muscle of a tongue in accordance with the present disclosure.

FIG. 5E depicts exercises targeting a hyoglossus muscle of a tongue in accordance with the present disclosure.

FIG. 5F depicts exercises targeting a hyoglossus muscle of a tongue in accordance with the present disclosure.

FIG. 6A depicts a palatoglossus muscle of a tongue.

FIG. 6B depicts a palatoglossus muscle of a tongue.

FIG. 6C depicts exercises targeting a palatoglossus muscle of a tongue in accordance with the present disclosure.

FIG. 6D depicts exercises targeting a palatoglossus muscle of a tongue in accordance with the present disclosure.

FIG. 6E depicts exercises targeting a palatoglossus muscle of a tongue in accordance with the present disclosure.

FIG. 6F depicts exercises targeting a palatoglossus muscle of a tongue in accordance with the present disclosure.

FIG. 6G depicts exercises targeting a palatoglossus muscle of a tongue in accordance with the present disclosure.

FIG. 6H depicts exercises targeting a palatoglossus muscle of a tongue in accordance with the present disclosure.

FIG. 6I depicts exercises targeting a palatoglossus muscle of a tongue in accordance with the present disclosure.

FIG. 6J depicts exercises targeting a palatoglossus muscle of a tongue in accordance with the present disclosure.

FIG. 6K depicts exercises targeting a palatoglossus muscle of a tongue in accordance with the present disclosure.

FIG. 7A depicts a lateral-pterygoid muscle of a head.

FIG. 7B depicts exercises targeting a lateral-pterygoid muscle of a head in accordance with the present disclosure.

FIG. 7C depicts exercises targeting a lateral-pterygoid muscle of a head in accordance with the present disclosure.

FIG. 7D depicts exercises targeting a lateral-pterygoid muscle of a head in accordance with the present disclosure.

FIG. 8A depicts a masseter muscle of a head.

FIG. 8B depicts exercises targeting a masseter muscle of a head in accordance with the present disclosure.

FIG. 8C depicts exercises targeting a masseter muscle of a head in accordance with the present disclosure.

FIG. 8D depicts exercises targeting a masseter muscle of a head in accordance with the present disclosure.

FIG. 8E depicts exercises targeting a masseter muscle of a head in accordance with the present disclosure.

FIG. 9A depicts a platysma muscle of a head and neck.

FIG. 9B depicts exercises targeting a platysma muscle of a head and neck in accordance with the present disclosure.

FIG. 9C depicts exercises targeting a platysma muscle of a head and neck in accordance with the present disclosure.

FIG. 9D depicts exercises targeting a platysma muscle of a head and neck in accordance with the present disclosure.

FIG. 9E depicts exercises targeting a platysma muscle of a head and neck in accordance with the present disclosure.

FIG. 10A depicts an orbicularis muscle of a head.

FIG. 10B depicts exercises targeting an orbicularis muscle of the head and neck in accordance with the present disclosure.

FIG. 10C depicts exercises targeting an orbicularis muscle of the head and neck in accordance with the present disclosure.

FIG. 10D depicts exercises targeting an orbicularis muscle of the head and neck in accordance with the present disclosure.

FIG. 10E depicts exercises targeting an orbicularis muscle of the head and neck in accordance with the present disclosure.

FIG. 11A depicts a levator labii superioris muscle of a head.

FIG. 11B depicts a risorius muscle of a head.

FIG. 11C depicts exercises targeting a levator labii superioris muscle of a head and a risorius muscle of a head in accordance with the present disclosure.

FIG. 11D depicts exercises targeting a levator labii superioris muscle of a head and a risorius muscle of a head in accordance with the present disclosure.

FIG. 11E depicts exercises targeting a levator labii superioris muscle of a head and a risorius muscle of a head in accordance with the present disclosure.

FIG. 11F depicts exercises targeting a levator labii superioris muscle of a head and a risorius muscle of a head in accordance with the present disclosure.

FIG. 12A depicts a zygomaticus major muscle of a head.

FIG. 12B depicts a zygomaticus minor muscle of a head.

FIG. 12C depicts exercises targeting a zygomaticus major muscle of a head and a zygomaticus minor muscle of a head in accordance with the present disclosure.

FIG. 12D depicts exercises targeting a zygomaticus major muscle of a head and a zygomaticus minor muscle of a head in accordance with the present disclosure.

FIG. 12E depicts exercises targeting a zygomaticus major muscle of a head and a zygomaticus minor muscle of a head in accordance with the present disclosure.

FIG. 12F depicts exercises targeting a zygomaticus major muscle of a head and a zygomaticus minor muscle of a head in accordance with the present disclosure.

FIG. 13A depicts a system including tools configured to be used for performing orofacial myofunctional exercises in accordance with the present disclosure.

FIG. 13B depicts tools configured to be used for performing orofacial myofunctional exercises in accordance with the present disclosure.

FIG. 13C depicts tools configured to be used for performing orofacial myofunctional exercises in accordance with the present disclosure.

FIG. 13D depicts tools configured to be used for performing orofacial myofunctional exercises in accordance with the present disclosure.

FIG. 13E depicts tools configured to be used for performing orofacial myofunctional exercises in accordance with the present disclosure.

FIG. 13F depicts tools configured to be used for performing orofacial myofunctional exercises in accordance with the present disclosure.

FIG. 13G depicts tools configured to be used for performing orofacial myofunctional exercises in accordance with the present disclosure.

FIG. 13H depicts tools configured to be used for performing orofacial myofunctional exercises in accordance with the present disclosure.

FIG. 13I depicts tools configured to be used for performing orofacial myofunctional exercises in accordance with the present disclosure.

FIG. 14A depicts a depressor anguli oris muscle of a head.

FIG. 14B depicts a depressor labii inferioris muscle of a head.

FIG. 14C depicts a mentalist muscle of a head.

FIG. 14D depicts exercises targeting a depressor anguli oris muscle of a head, a depressor labii inferioris muscle of the head, and a mentalist muscle of the head in accordance with the present disclosure.

FIG. 14E depicts exercises targeting a depressor anguli oris muscle of a head, a depressor labii inferioris muscle of the head, and a mentalist muscle of the head in accordance with the present disclosure.

FIG. 14F depicts exercises targeting a depressor anguli oris muscle of a head, a depressor labii inferioris muscle of the head, and a mentalist muscle of the head in accordance with the present disclosure.

FIG. 14G depicts exercises targeting a depressor anguli oris muscle of a head, a depressor labii inferioris muscle of the head, and a mentalist muscle of the head in accordance with the present disclosure.

FIG. 14H depicts exercises targeting a depressor anguli oris muscle of a head, a depressor labii inferioris muscle of the head, and a mentalist muscle of the head in accordance with the present disclosure.

FIG. 14I depicts exercises targeting a depressor anguli oris muscle of a head, a depressor labii inferioris muscle of the head, and a mentalist muscle of the head in accordance with the present disclosure.

FIG. 15A depicts a posture of a head.

FIG. 15B depicts exercises targeting an improved posture of a head in accordance with the present disclosure.

FIG. 15C depicts exercises targeting an improved posture of a head in accordance with the present disclosure.

FIG. 15D depicts exercises targeting an improved posture of a head in accordance with the present disclosure.

FIG. 15E depicts exercises targeting an improved posture of a head in accordance with the present disclosure.

FIG. 16A depicts exercises targeting improved nasal breathing in accordance with the present disclosure.

FIG. 16B depicts exercises targeting improved nasal breathing in accordance with the present disclosure.

FIG. 16C depicts exercises targeting improved nasal breathing in accordance with the present disclosure.

FIG. 16D depicts exercises targeting improved nasal breathing in accordance with the present disclosure.

FIG. 16E depicts exercises targeting improved nasal breathing in accordance with the present disclosure.

FIG. 16F depicts exercises targeting improved nasal breathing in accordance with the present disclosure.

FIG. 16G depicts exercises targeting improved nasal breathing in accordance with the present disclosure.

FIG. 16H depicts exercises targeting improved nasal breathing in accordance with the present disclosure.

FIG. 17A depicts a posture of a head.

FIG. 17B depicts exercises targeting an improved posture of a head in accordance with the present disclosure.

FIG. 17C depicts exercises targeting an improved posture of a head in accordance with the present disclosure.

FIG. 17D depicts exercises targeting an improved posture of a head in accordance with the present disclosure.

FIG. 18 depicts data indicating improved clinical outcomes of oral appliances combined with orofacial myofunctional therapy in accordance with the present disclosure.

FIG. 19 depicts an orofacial myofunctional therapy program in accordance with the present disclosure.

FIG. 20 depicts a tracking system and procedure for an orofacial myofunctional therapy program in accordance with the present disclosure.

FIG. 21 depicts an awareness method for an orofacial myofunctional therapy program in accordance with the present disclosure.

FIG. 22 is a block diagram of an exemplary computer system in accordance with the present disclosure.

FIG. 23 is an exemplary configuration of a server system in accordance with the present disclosure.

FIG. 24 is an exemplary configuration of a client system in accordance with the present disclosure.

### DETAILED DESCRIPTION OF THE DISCLOSURE

The methods and systems of the present disclosure combine orofacial myofunctional therapy exercises with an oral appliance to non-surgically treat OMDs. Oral appliances have proven successful in non-surgically treating OMDs, including orthodontic, dentofacial, and/or craniofacial variations. By using a method that combines the use of oral appliances with orofacial myofunctional therapy, patients being treated for OMDs may experience improved clinical outcomes.

### Methods.

Generally, the method described herein treats an orofacial muscle disorder in a patient. In some embodiments, the method includes: (i) modifying a first component of an oral anatomy of the patient with an oral appliance; and (ii) exercising a second component of the oral anatomy of the patient with at least one orofacial myofunctional therapy exercise.

As used herein, a “patient” refers to a human patient. The human patient may be a child or an adult. For brevity, when the term “patient” is used herein, the term may also refer to a guardian of the patient. The guardian may be a parent or legal guardian of the patient.

Generally, the first component of the oral anatomy of the patient may be any suitable component of the oral anatomy of the patient that advances the methods described herein. In some embodiments, the first component of the oral anatomy of the patient is selected from the group consisting of an airway, an airway including a nasal cavity, an oral cavity, and an upper and lower airway, an upper airway, a tooth position, a jaw position, an upper jaw position, a lower jaw position, a tongue position, a lip position, a bite, a component relating to breathing, a component relating to posture, a component relating to speech, a component relating to sleep disordered breathing symptoms, and combinations thereof. As used herein, an airway may include one or more airways, including a nasal cavity, an oral cavity, an upper airway, and combinations thereof. In some embodiments, the first component of the oral anatomy of the patient includes an upper airway.

In some embodiments, modifying the first component of the oral anatomy of the patient includes increasing the size of the airway of the patient and/or changing the shape of the airway of the patient. In some embodiments, the method increases the size of the airway of the patient and/or changes the shape of the airway of the patient, and further trains the patient on how to use the increased the size of the airway and/or the changed shape of the airway.

Generally, the oral appliance and the at least one orofacial myofunctional therapy exercise are combined to provide improved benefits. In many embodiments, the oral appliance and the at least one orofacial myofunctional therapy exercise are combined at least once. In some embodiments, the oral appliance and the at least one orofacial myofunctional therapy exercise are combined more than once. In some embodiments, the oral appliance and the at least one orofacial myofunctional therapy exercise are combined more than once over a time period selected from the group consisting of hours, days, weeks, months, and years.

In some embodiments, the first component of the oral anatomy of the patient is modified before, concurrently with, and/or after the second component of the oral anatomy of the patient is exercised.

In some embodiments, exercising the second component of the oral anatomy of the patient includes at least one, at least two, at least three, at least four, at least five, at least six, at least seven, at least eight, at least nine, at least ten, or more than ten orofacial myofunctional therapy exercises.

In some embodiments, exercising the second component of the oral anatomy of the patient includes less than eleven, less than ten, less than nine, less than eight, less than seven, less than six, less than five, less than four, less than three, or less than two orofacial myofunctional therapy exercises.

In some embodiments, exercising the second component of the oral anatomy of the patient includes using at least one tool to perform the at least one orofacial myofunctional therapy exercise. Generally, the at least one tool may include any suitable tool that advances the methods described herein.

In some embodiments, exercising the second component of the oral anatomy of the patient includes exercising the second component of the oral anatomy of the patient with at least one orofacial myofunctional therapy exercise in a multi-phase orofacial myofunctional therapy program.

In some embodiments, the second component of the oral anatomy of the patient is selected from the group consisting of head posture, resting tongue position, tongue strength, breathing patterns, nasal breathing, lip seal, orofacial muscles, chewing and swallowing patterns, oral noxious habits, functional frenuloplasty, range of motion, tongue range of motion, elasticity in facial muscles, speech, psychological associations thereof, and combinations thereof.

In some embodiments, the second component of the oral anatomy of the patient is selected from the group consisting of head muscles, head airways, face muscles, face tissues, nasal muscles, nasal tissues, tongue muscles, tongue tissues, throat muscles, throat tissues, lip muscles, lip tissues, cheek muscles, cheek tissues, jaw muscles, jaw tissues, and combinations thereof.

In some embodiments, the second component of the oral anatomy of the patient is selected from the group consisting of orofacial muscles, cranial muscles, neck muscles, a genioglossus muscle, a styloglossus muscle, a hyoglossus muscle, a palatoglossus muscle, a lateral-pterygoid muscle, a masseter muscle, a superficial masseter muscle, a platysma muscle, an orbicularis muscle, a levator labii superioris muscle, a risorius muscle, a zygomaticus major muscle, a zygomaticus minor muscle, a zygomaticus muscle, a depressor anguli oris muscle, a depressor labii inferioris muscle, a mentalist muscle, a superior lingualis muscle, an inferior lingualis muscle, a vertical lingualis muscle, a transverse lingualis muscle, a buccinator muscle, and combinations thereof.

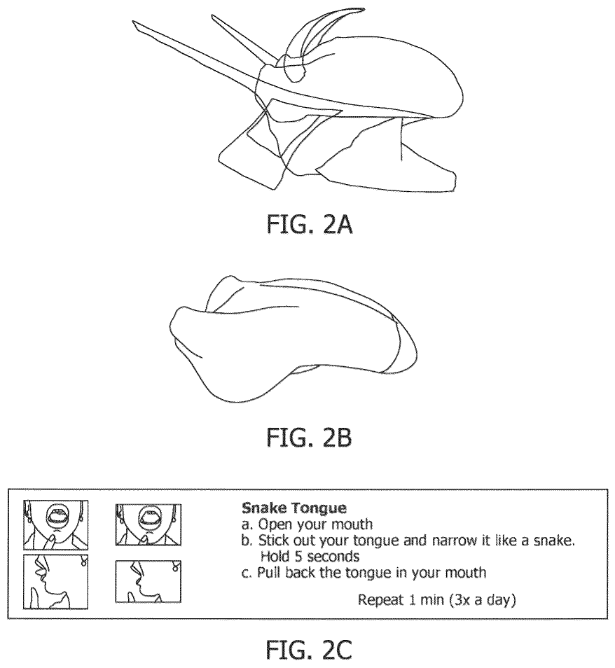

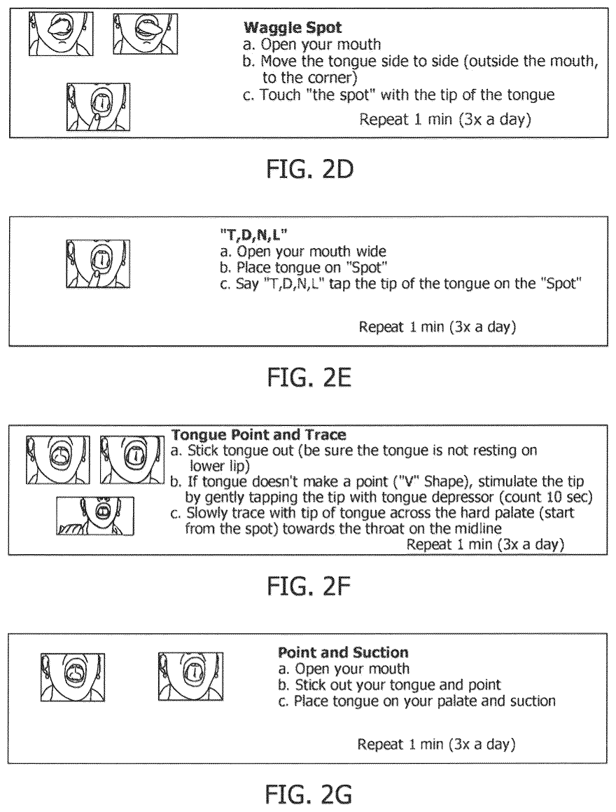

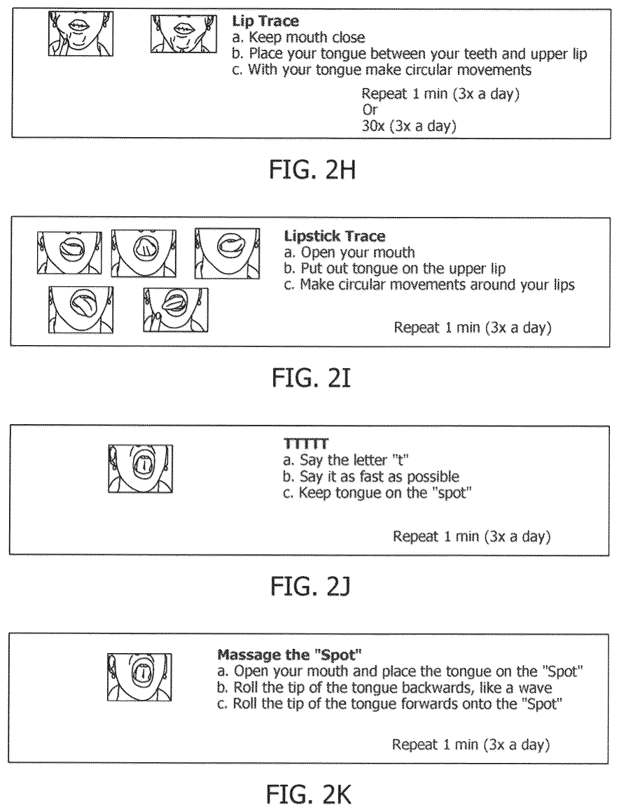

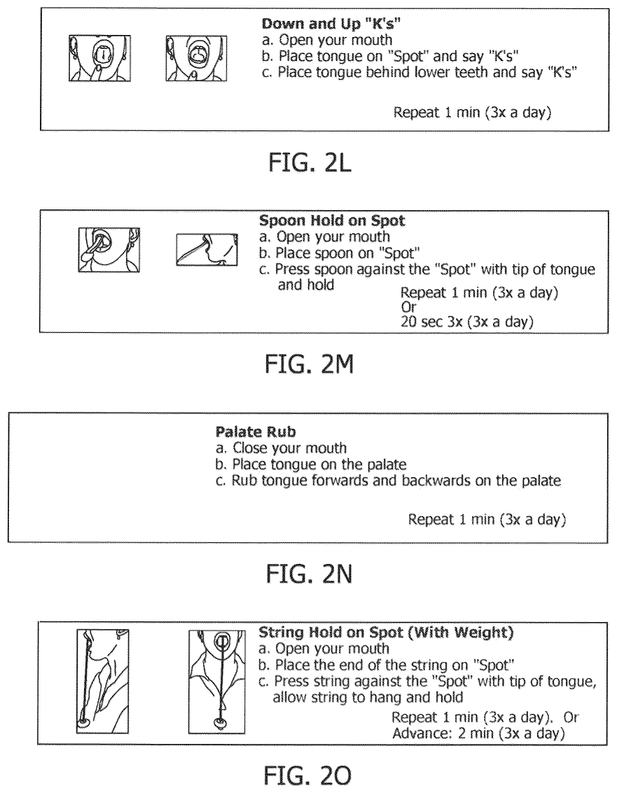

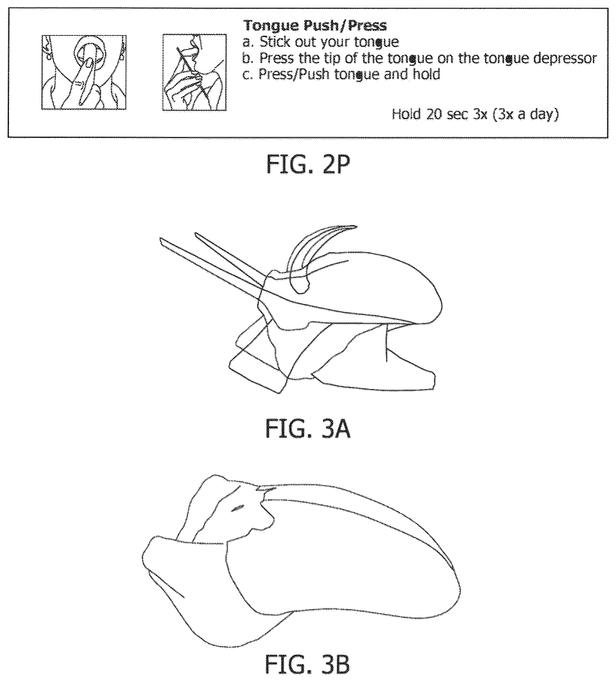

In some embodiments, the method includes at least one awareness exercise to increase a patient's awareness of their lingual oral space. Exemplary awareness exercises are illustrated in FIG. 2I. In some embodiments, these exercises increase a patient's lingual oral space awareness through neuroplasticity. In these embodiments, the awareness exercise strengthens one or more psychological association between an oral noxious habit and a physical component of the oral anatomy of the patient.

In some embodiments, the method further includes providing the patient with the oral appliance. In some embodiments, the method further includes providing the patient with instructions to perform the at least one orofacial myofunctional therapy exercise.

### Orofacial Myofunctional Therapy.

Generally, the methods and systems of the present disclosure may be used with any suitable orofacial myofunctional therapy exercise known in the art. Suitable orofacial myofunctional therapy exercises may be selected based on a patient evaluation by a healthcare professional, such as a therapist. In some embodiments, orofacial myofunctional therapy exercises target the muscles and the soft tissues of the upper airway to keep the upper airway unobstructed in daytime and during sleep.

Orofacial myofunctional therapy including orofacial myofunctional therapy exercises combined with an oral appliance has been demonstrated herein to improve the clinical outcome of the oral appliances (illustrated by FIG. 18). It has also been previously shown that myofunctional therapy alone may reduce the apnea hypopnea index (AHI) in adults by up to 50% and in children by up to 65% (Camacho et al., “Myofunctional Therapy to Treat Obstructive Sleep Apnea: A Systematic Review and Meta-analysis”, Sleep, Vol. 38, No. 5, 2015). Such benefits to AHI are achievable as well with the methods according to the present disclosure.

In some embodiments, at least one exercise is completed in the presence of a healthcare professional. The healthcare professional may be, for example, a therapist, an orthodontist, a dental hygienist, an orofacial myologist, a general dentist, a doctor, a nurse, an otorhinolaryngologist, or a speech-language pathologist. The presence of a healthcare professional may be a physical presence and/or a virtual presence. In some embodiments, at least one exercise is completed in the absence of a healthcare professional.

In some embodiments, exercising the second component of the oral anatomy of the patient includes exercising the second component of the oral anatomy of the patient with at least one orofacial myofunctional therapy exercise in a multi-phase orofacial myofunctional therapy program.

In some embodiments, the orofacial myofunctional therapy includes an initial evaluation by a healthcare professional followed by three phases of therapy, including an intensive phase, a generalization/maintenance phase, and a habituation phase. The orofacial myofunctional therapy may be customized based on the patient's condition, which may be, for example, mild, standard, and/or severe. The patient's condition may require standard and/or supplemental therapy. The patient's condition may depend on an OSA score and the presence of atypical swallow, frenulum, and/or jaw pain/discomfort.

In some embodiments, the orofacial myofunctional therapy is in the form of a multi-phase therapy. In some embodiments, the orofacial myofunctional therapy includes at least one phase, at least two phases, at least three phases, at least four phases, or at least five phases.

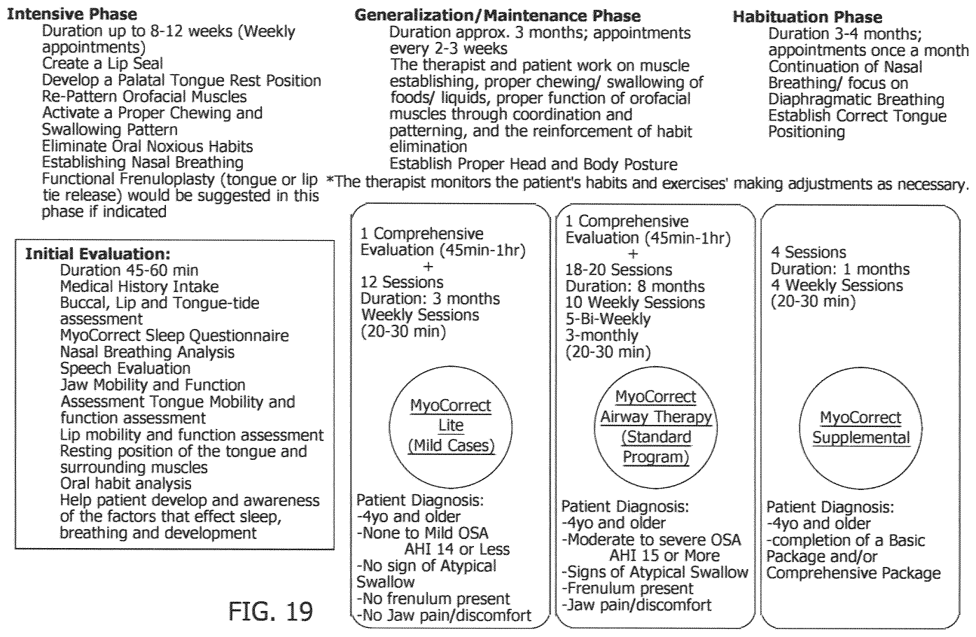

In some embodiments, the orofacial myofunctional therapy is in the form of a three-phase therapy. An exemplary embodiment of a three-phase therapy is shown in FIG. 19.

Generally, the phases of the orofacial myofunctional therapy may include an intensive phase, a generalization/maintenance phase, and/or a habituation phase. Each phase may be associated with the completion of different exercises specific to that phase.

In some embodiments, the intensive phase may be a duration of 8-12 weeks, including weekly appointments with a therapist, and may include exercises targeted to: creating a lip seal, developing a palatal tongue rest position, re-pattern orofacial muscles, activating a proper chewing and swallowing pattern, eliminating oral noxious habits, establishing nasal breathing, and/or functional frenuloplasty.

In some embodiments, the selection of an orofacial myofunctional therapy exercise may be based in part on achieving a specific outcome for the patient, including, but not limited to: correction of tongue placement, elimination of bad oral habits, nasal decongestion, jaw stabilization, increase of strength, and/or correction of function.

In some embodiments, the generalization/maintenance phase has a duration of about 3 months, includes appointments every 2-3 weeks with a therapist, and may include exercises targeted to: establish muscle, establish proper chewing and/or swallowing of foods and/or liquids, establish proper function of orofacial muscles through coordination and patterning, reinforce habit elimination, and/or establish proper head and body posture.

In some embodiments, the habituation phase has a duration of about 3-4 months, includes monthly appointments with a therapist, and may include exercises targeted to: continue nasal breathing, focus on diaphragmatic breathing, and/or establish correct tongue positioning.

Preferred orofacial myofunctional therapy exercises are shown in the figures of the present disclosure. These preferred exercises are described in detail herein.

### Resting Tongue Position.

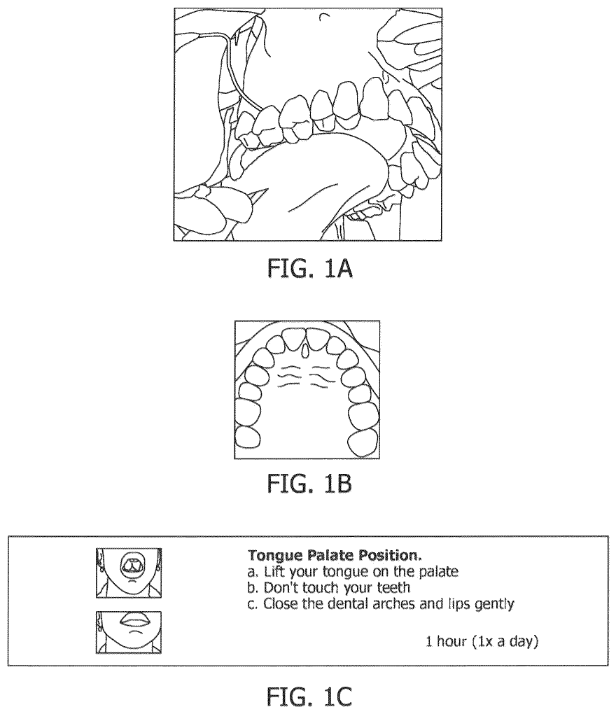

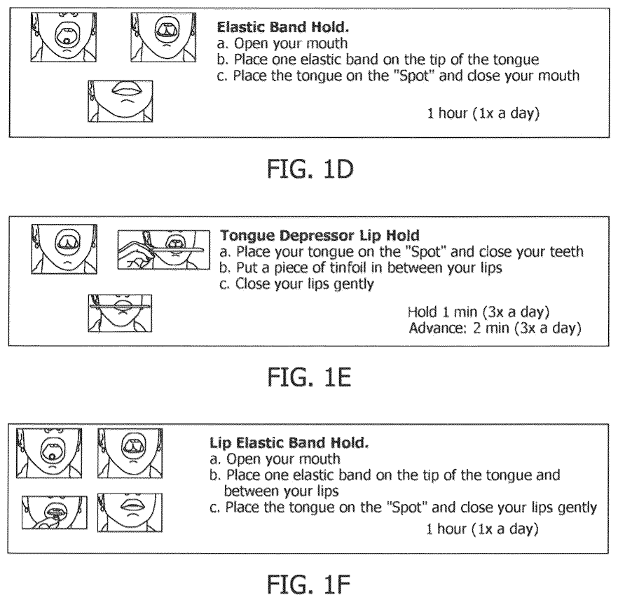

Orofacial myofunctional therapy exercises targeting the resting tongue position (illustrated by FIG. 1A and FIG. 1B) may help correct the tongue placement of a patient. The apex of the tongue should rest on the incisive papilla and all parts of the tongue should touch the whole palate and suction itself at all times when resting. Exercises targeting the resting tongue position include: tongue palate position (FIG. 1C), elastic band hold (FIG. 1D), tongue depressor lip hold (FIG. 1E), and lip elastic band hold (FIG. 1F).

### Increase Tongue Strength.

Orofacial myofunctional therapy exercises targeting an increase of tongue strength may help the four intrinsic muscles of the tongue (the superior lingualis, the inferior lingualis, the vertical lingualis, and the transverse lingualis) to work together to increase tongue flexibility, as well as help the four extrinsic muscles of the tongue that originate from structures other than the tongue to work together (illustrated by FIG. 2A and FIG. 2B). Exercises targeting an increase of tongue strength include: snake tongue (FIG. 2C), waggle spot (FIG. 2D), “T,D,N,L” (FIG. 2E), tongue point and trace (FIG. 2F), point and sunction (FIG. 2G), lip trace (FIG. 2H), lipstick trace (FIG. 2I), TTTTT (FIG. 2J), massage the “spot” (FIG. 2K), down and up “k's” (FIG. 2L), spoon hold on spot (FIG. 2M), palate rub (FIG. 2N), string hold on spot (with weight) (FIG. 2O), and tongue push/press (FIG. 2P).

### Genioglossus.

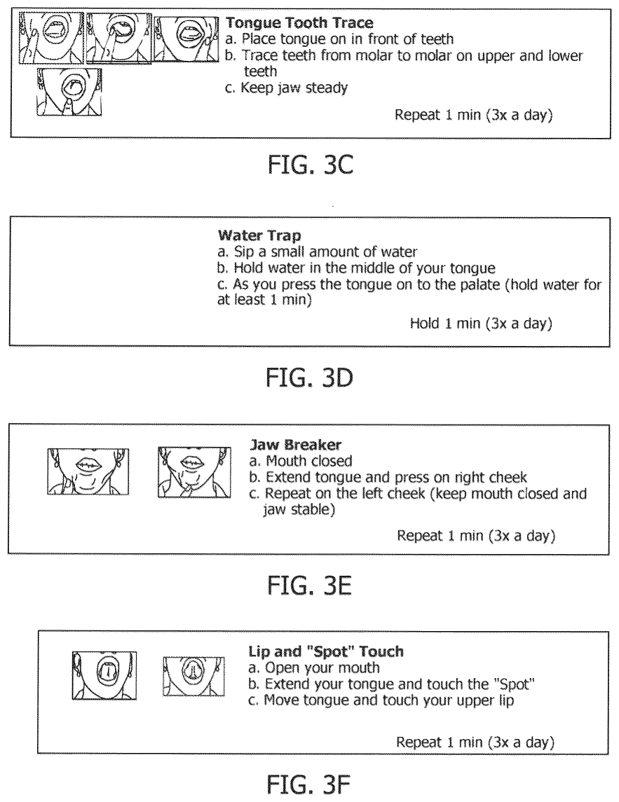

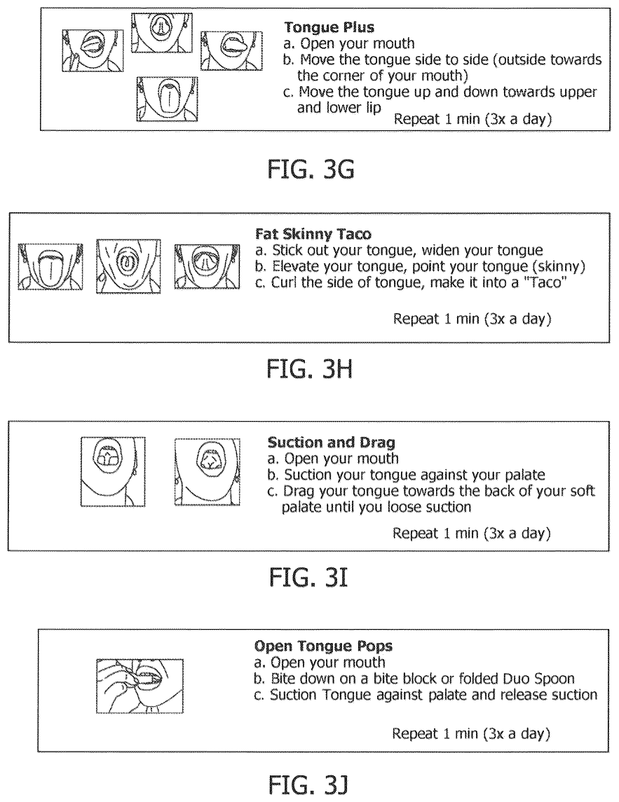

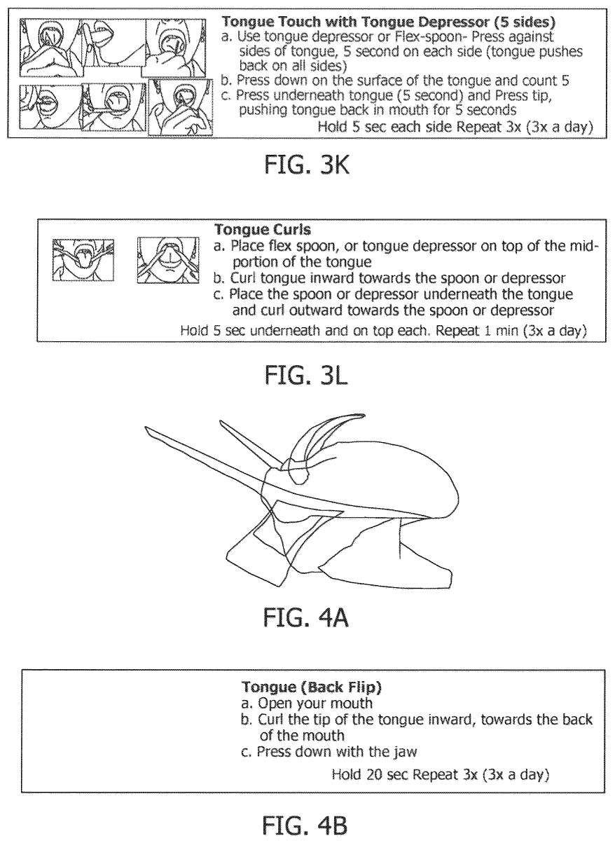

Orofacial myofunctional therapy exercises targeting the genioglossus (illustrated by FIG. 3A and FIG. 3B), a muscle of the tongue that originates from the inferior mental spine of the mandible, may help depress and extend the tongue. Exercises targeting the genioglossus include: tongue tooth trace (FIG. 3C), water trap (FIG. 3D), jaw breaker (FIG. 3E), lip and “spot” touch (FIG. 3F), tongue plus (FIG. 3G), fat skinny taco (FIG. 3H), suction and drag (FIG. 3I), open tongue pops (FIG. 3J), tongue touch with tongue depressor (5 sides) (FIG. 3K), and tongue curls (FIG. 3L).

### Styloglossus.

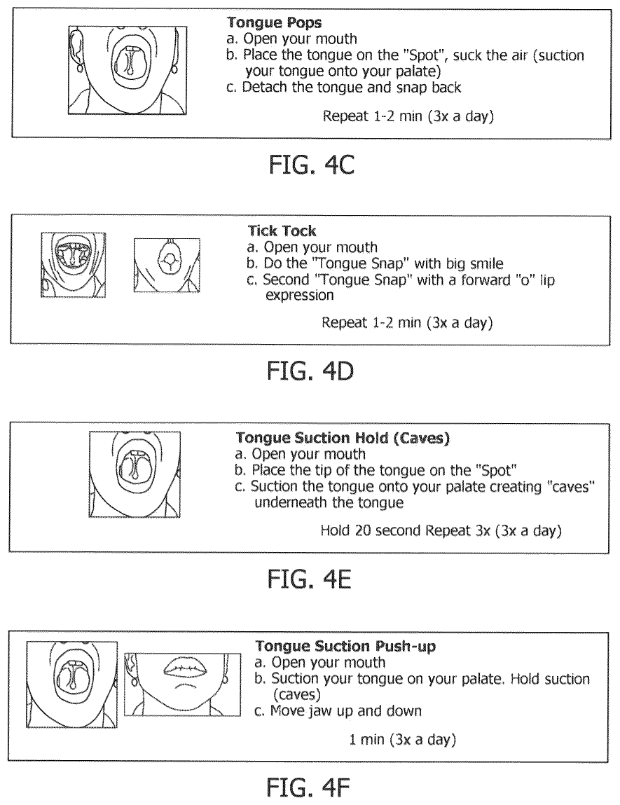

Orofacial myofunctional therapy exercises targeting the styloglossus (illustrated by FIG. 4A), an extrinsic muscle of the tongue that originates from the anterior lateral styloid process of the temporal bone, may help draw the sides of the tongue upward and draw the tongue back. Exercises targeting the styloglossus include: tongue (back flip) (FIG. 4B), tongue pops (FIG. 4C), tick took (FIG. 4D), tongue suction hold (caves) (FIG. 4E), and tongue suction push-up (FIG. 4F).

### Hyoglossus.

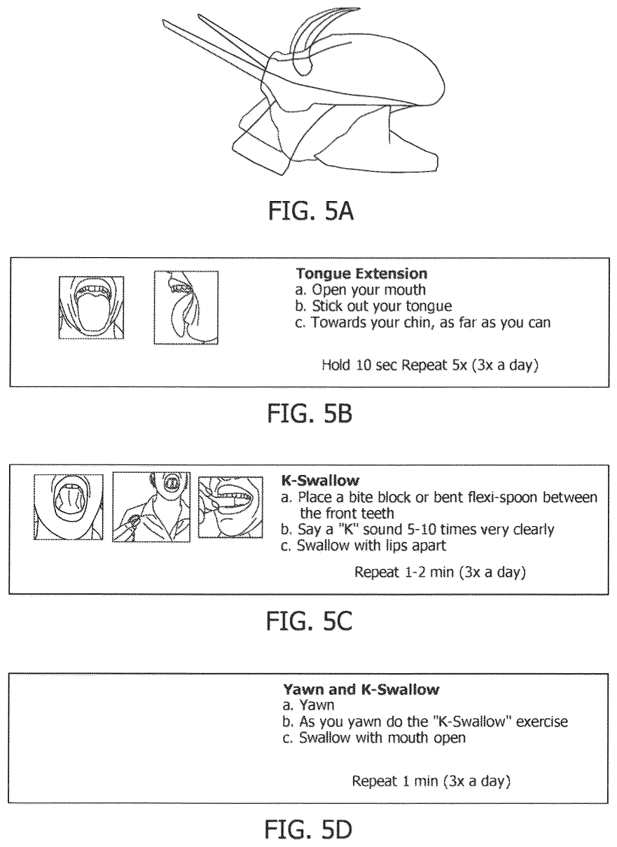

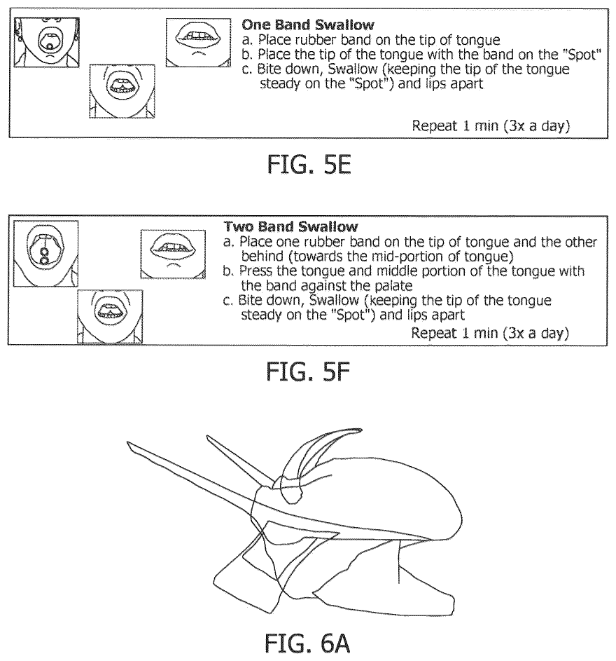

Orofacial myofunctional therapy exercises targeting the hyoglossus (illustrated by FIG. 5A), an extrinsic muscle of the tongue that originates from the greater cornu of the hyoid bone, may help depress the tongue and aid in swallowing. Exercises targeting the hyoglossus include: tongue extension (FIG. 5B), k-swallow (FIG. 5C), yawn and k-swallow (FIG. 5D), one band swallow (FIG. 5E), and two band swallow (FIG. 5F).

### Palatoglossus.

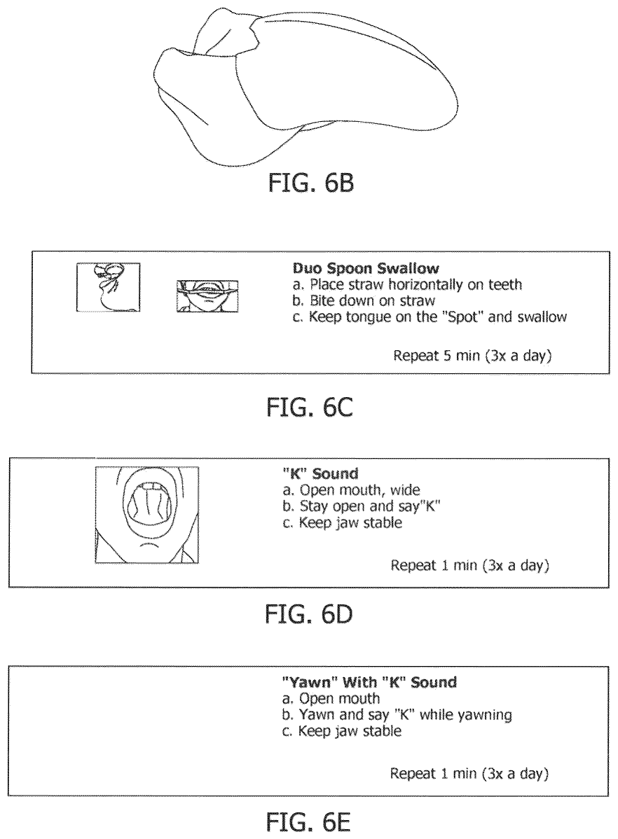

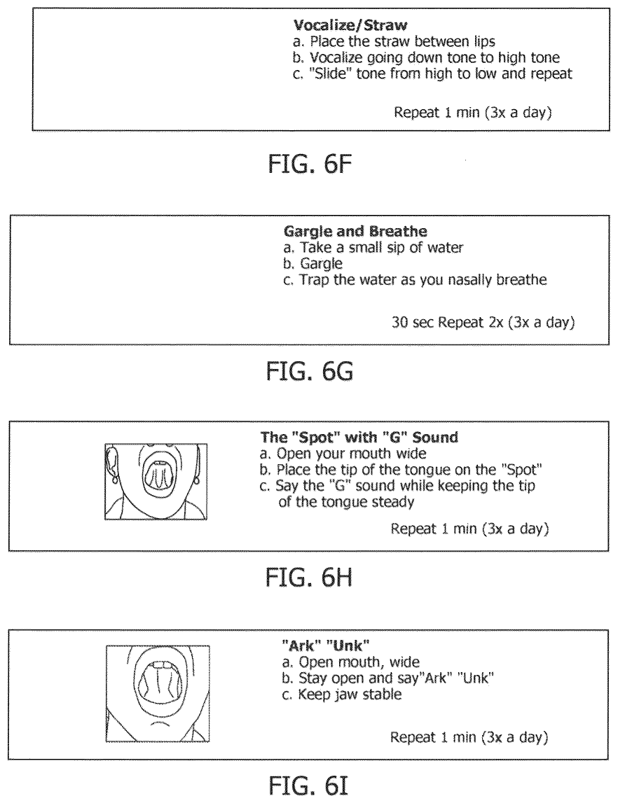

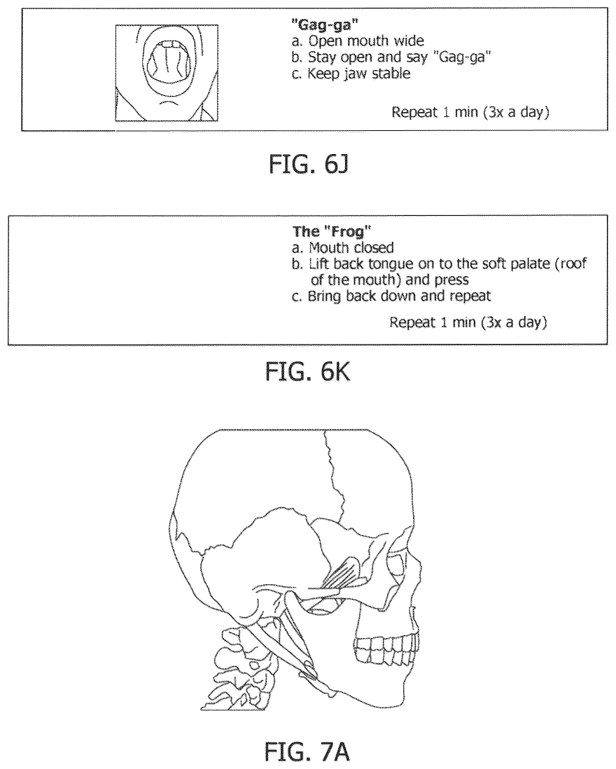

Orofacial myofunctional therapy exercises targeting the palatoglossus (illustrated by FIG. 6A and FIG. 6B), an extrinsic muscle of the tongue that originates from the anterior surface of the soft palate, may help elevate the use of the posterior thong and constrict the pharynx.

Exercises targeting the palatoglossus include: duo spoon swallow (FIG. 6C), “k” sound (FIG. 6D), “yawn” with “k” sound (FIG. 6E), vocalize/straw (FIG. 6F), gargle and breathe (FIG. 6G), the “spot” with “g” sound (FIG. 6H), “ark” “unk” (FIG. 6I), “gag-ga” (FIG. 6J), and the “frog” (FIG. 6K).

### Lateral-Pterygoid.

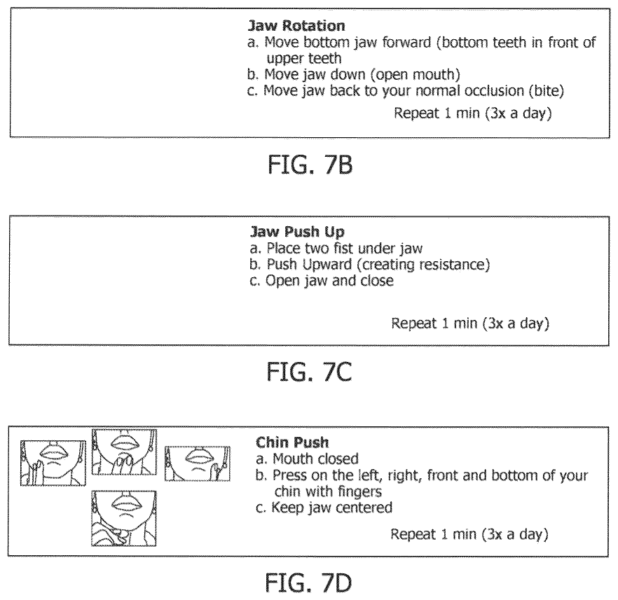

Orofacial myofunctional therapy exercises targeting the lateral-pterygoid (illustrated by FIG. 7A), a superficial muscle of mastication I, may help draw the mandibular condyle and articulation disc forward. Exercises targeting the lateral-pterygoid include: jaw rotation (FIG. 7B), jaw push up (FIG. 7C), and chin push (FIG. 7D).

### Superficial Masseter.

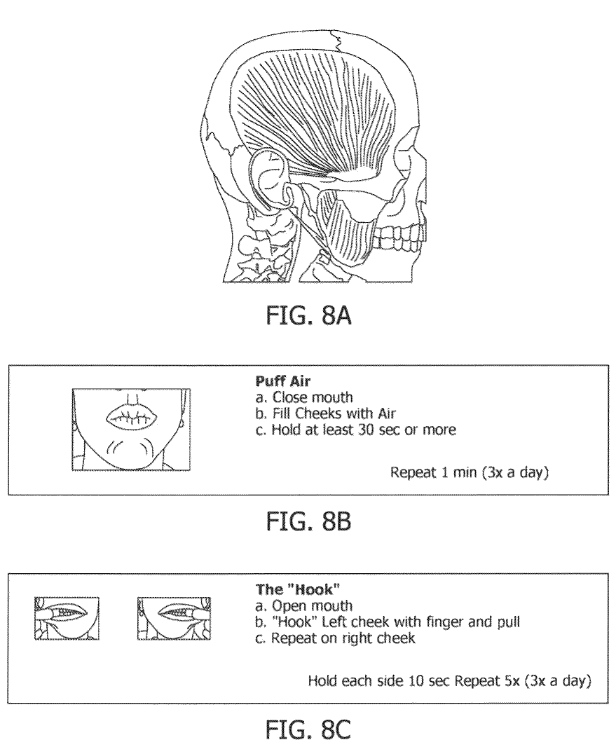

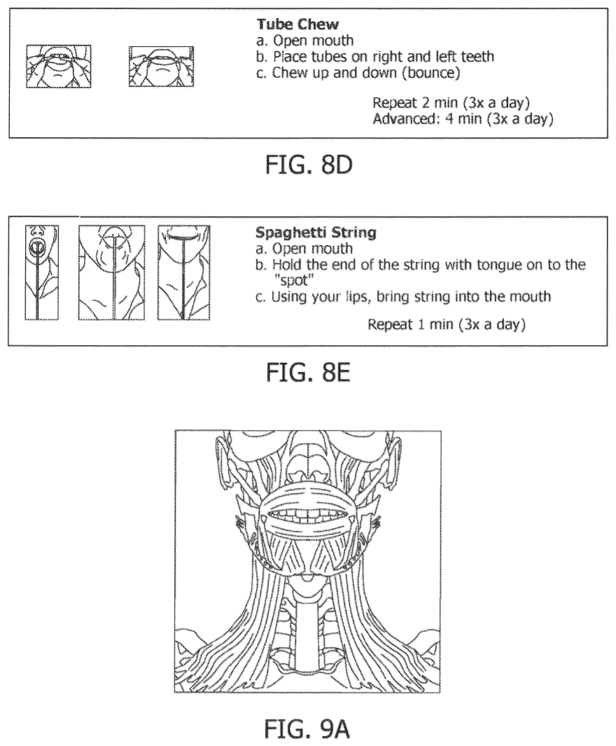

Orofacial myofunctional therapy exercises targeting the superficial masseter (illustrated by FIG. 8A), a superficial muscle of mastication, may help elevate and protract the mandible. Exercises targeting the superficial masseter include: puff air (FIG. 8B), the “hook” (FIG. 8C), tube chew (FIG. 8D), and spaghetti string (FIG. 8E).

### Platysma.

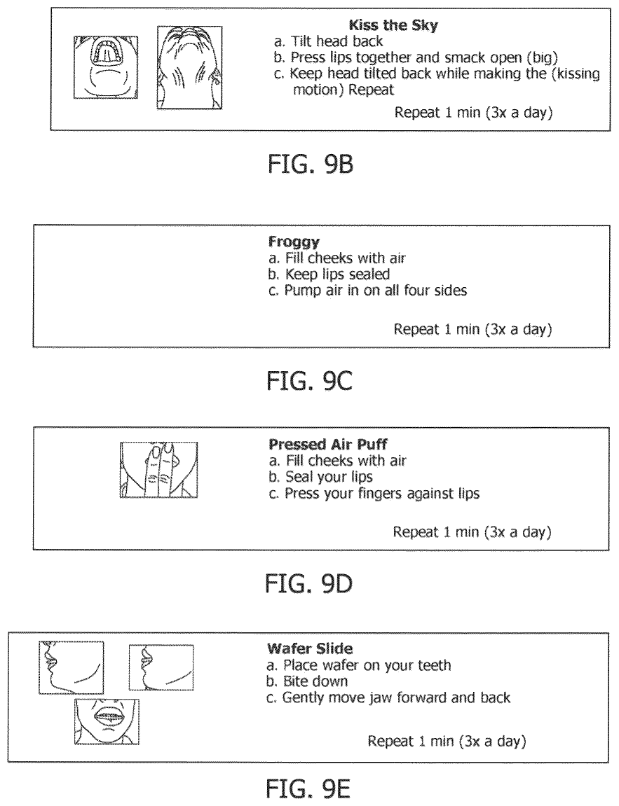

Orofacial myofunctional therapy exercises targeting the platysma (illustrated by FIG. 9A), a superficial muscle of facial expression and a neck muscle, may help depress the corners of the mouth and elect the neck skin when the teeth are clenched. Exercises targeting the platysma include: kiss the sky (FIG. 9B), froggy (FIG. 9C), pressed air puff (FIG. 9D), and wafer slide (FIG. 9E).

### Orbicularis.

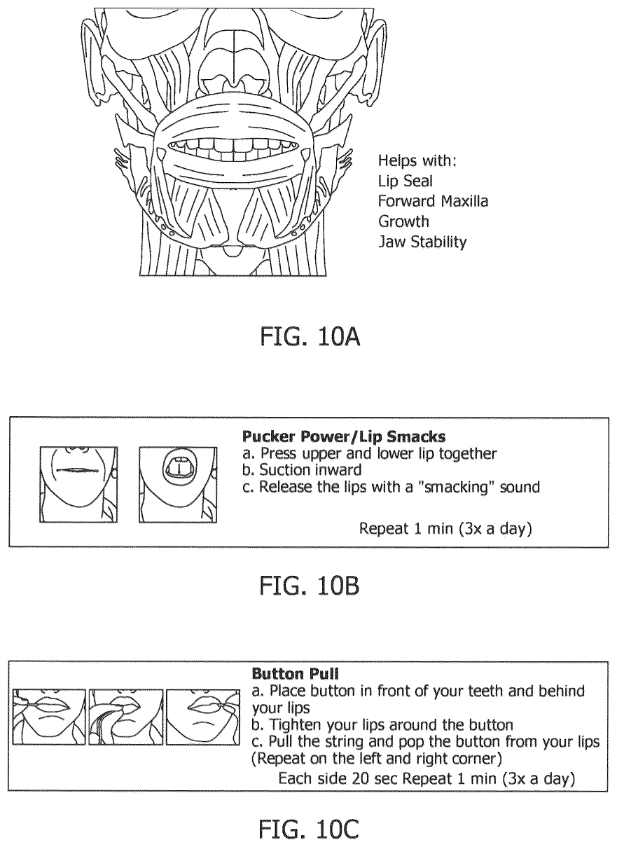

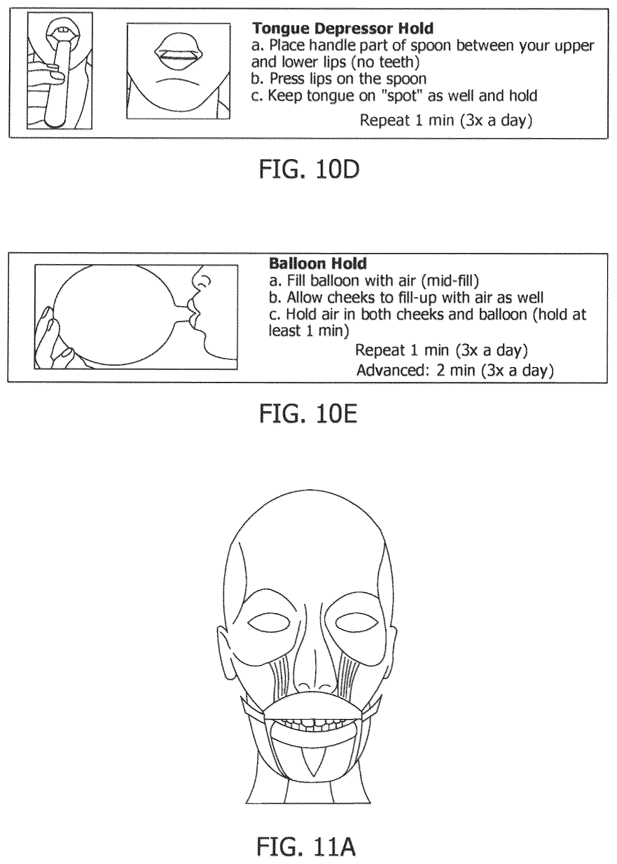

Orofacial myofunctional therapy exercises targeting the orbicularis (illustrated by FIG. 10A), the muscle that encircles the mouth, may help close the lips, protrude the lips, press the lips against the teeth, stabilize the jaw, and improve speech. Exercises targeting the orbicularis include pucker power/lip smacks (FIG. 10B), button pull (FIG. 10C), tongue depressor hold (FIG. 10D), and balloon hold (FIG. 10E).

### Levator Labii Superioris and Risorius.

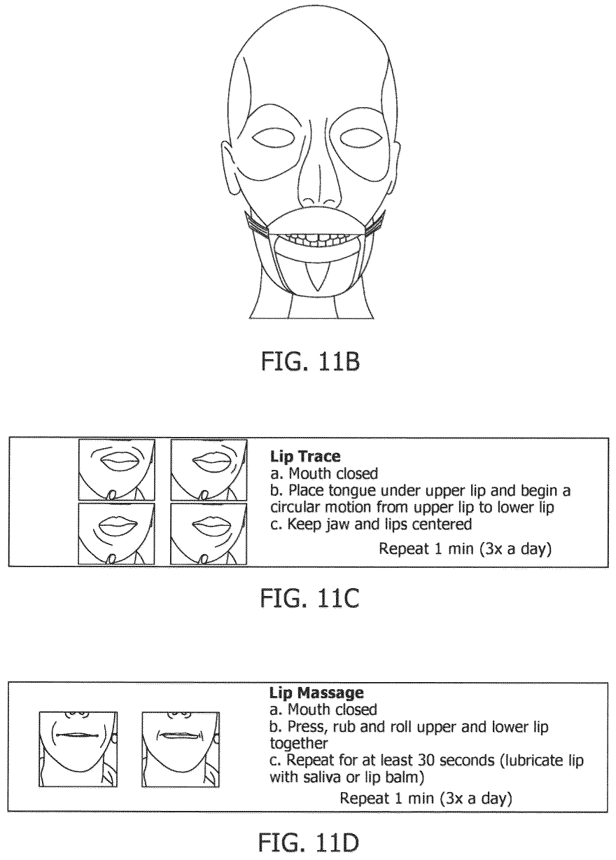

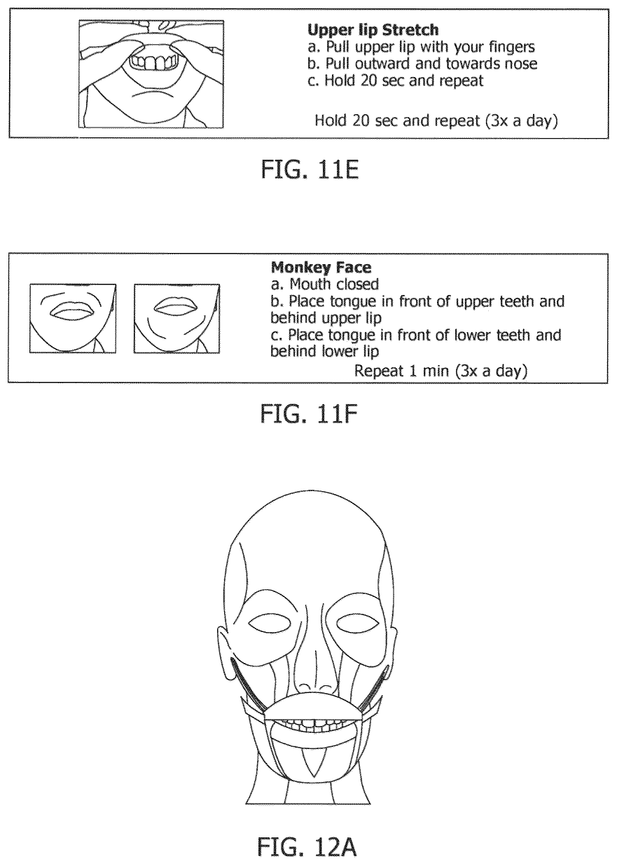

Orofacial myofunctional therapy exercises targeting the levator labii superioris and the risorius (illustrated by FIG. 1A and FIG. 11B), superficial muscles of facial expression and muscles of the mouth, may help raise and move forward the upper lip and retract the angle of the mouth. Exercises targeting the levator labii superioris and the risorius include: lip trace (FIG. 11C), lip massage (FIG. 11D), upper lip stretch (FIG. 11E), and monkey face (FIG. 11F).

### Zygomaticus Major and Zygomaticus Minor.

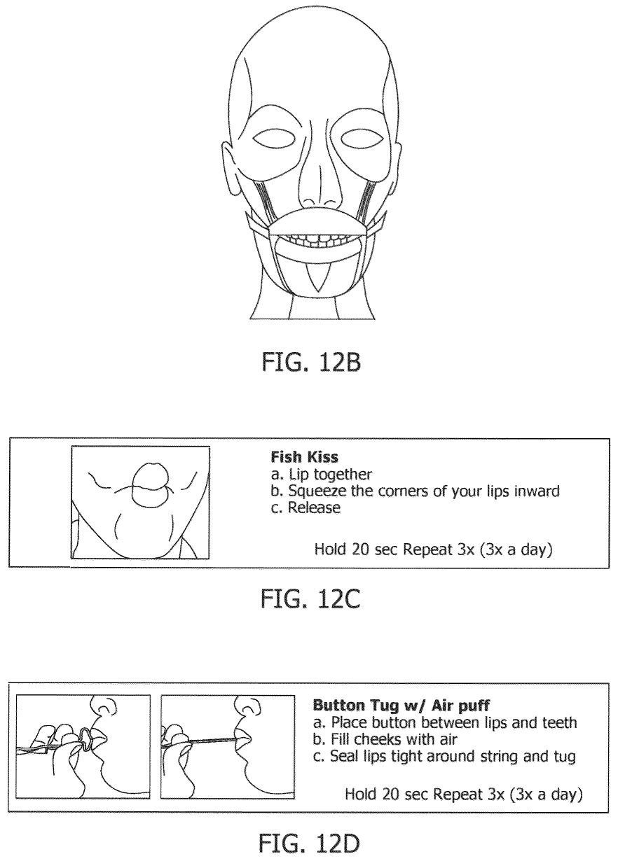

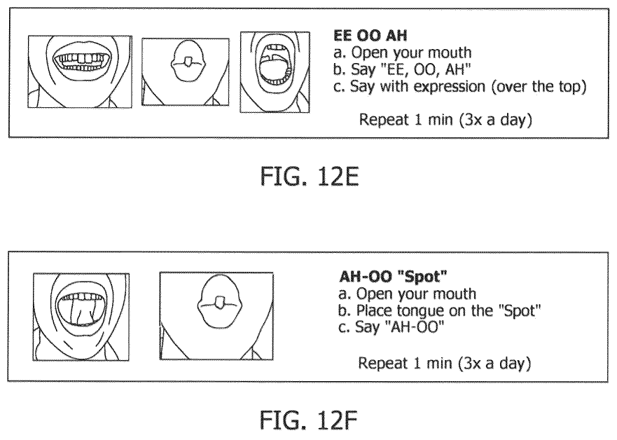

Orofacial myofunctional therapy exercises targeting the zygomaticus major and the zygomaticus minor (illustrated by FIG. 12A and FIG. 12B), superficial muscles of facial expression and muscles of the mouth, may help draw the angle of the mouth laterally and upward and draw the upper lip outward, upward, and laterally. Exercises targeting the zygomaticus major and the zygomaticus minor include: fish kiss (FIG. 12C), button tug with air puff (FIG. 12D), EE 00 AH (FIG. 12E), and AH-GO “spot” (FIG. 12F).

### Depressor Anguli Oris, Depressor Labii Inferioris, and Mentalist.

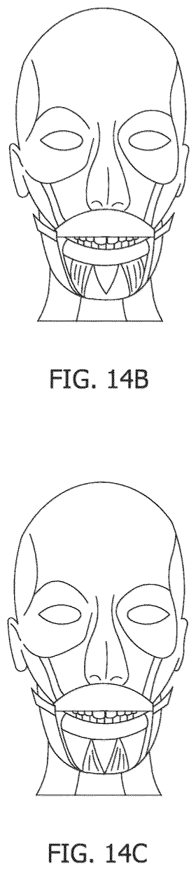

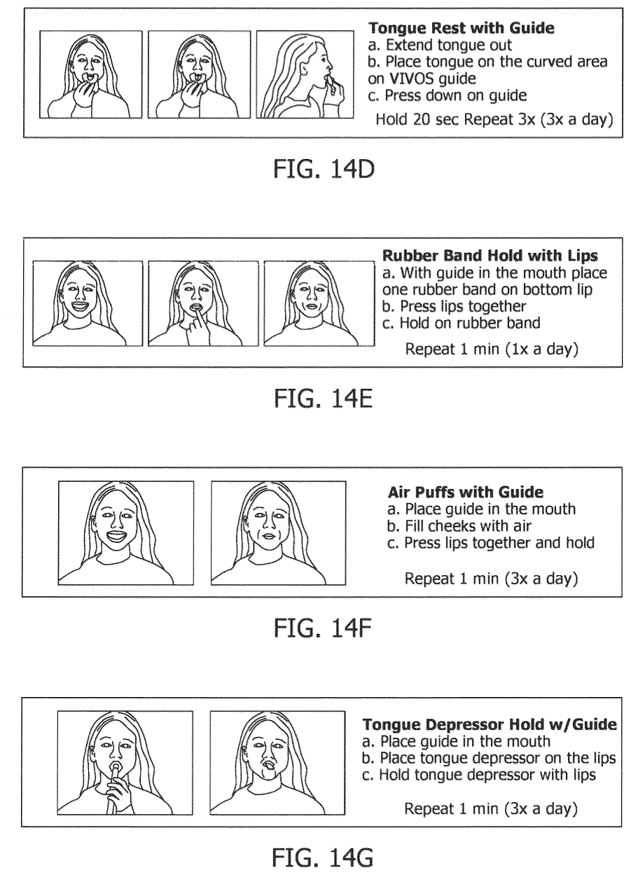

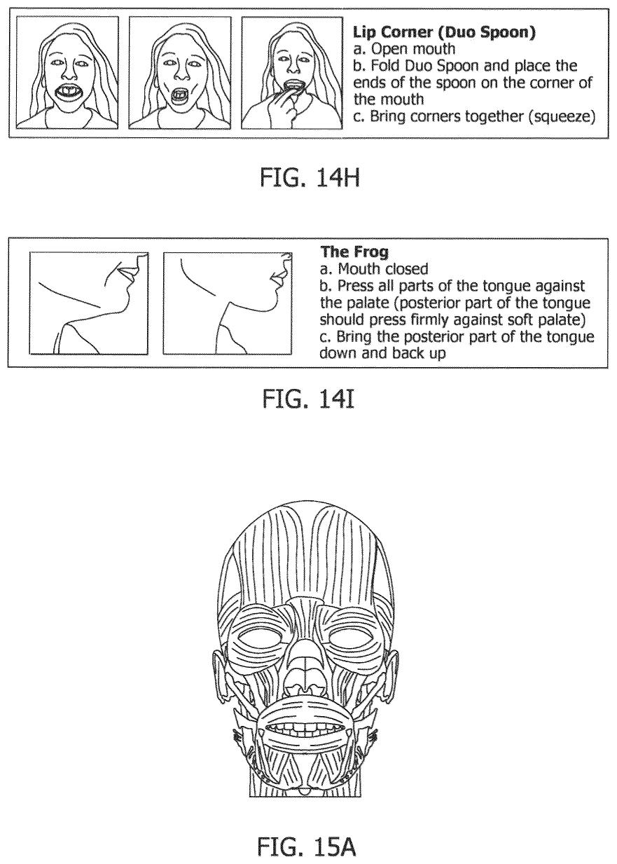

Orofacial myofunctional therapy exercises targeting the depressor anguli oris, the depressor labii inferioris, and the mentalist (FIG. 14A, FIG. 14B, and FIG. 14C), muscles of facial expression and the mouth, may help draw the angle of the mouth downward and laterally, draw the lower lip inferioris and laterally, raise and protrude the lower lip, and wrinkle the skin of the chin. Exercises targeting the depressor anguli oris, the depressor labii inferioris, and the mentalist include: tongue rest with guide (FIG. 14D), rubber band hold with lips (FIG. 14E), air puffs with guide (FIG. 14F), tongue depressor hold with guide (FIG. 14G), lip corner (duo spoon) (FIG. 14H), and the frog (FIG. 14I)

### Head Posture.

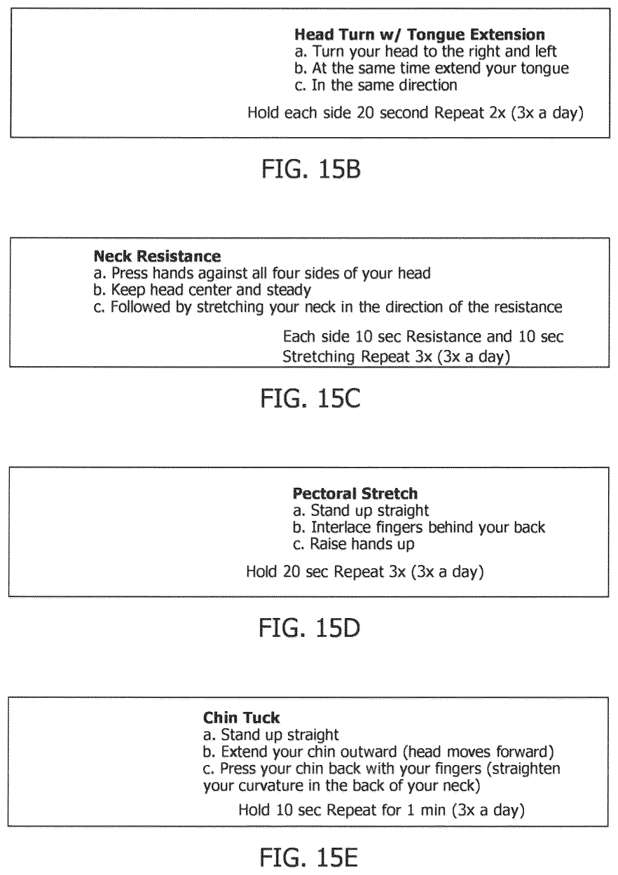

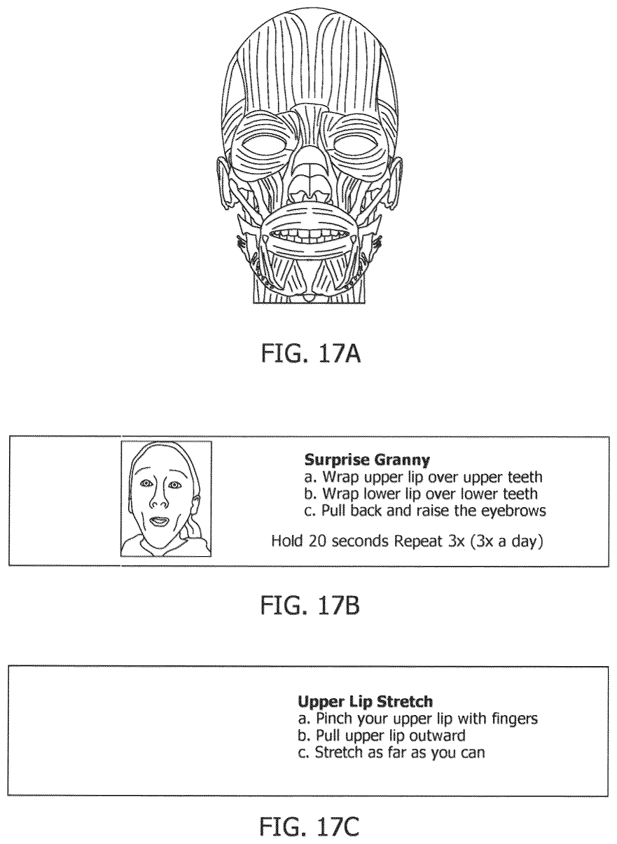

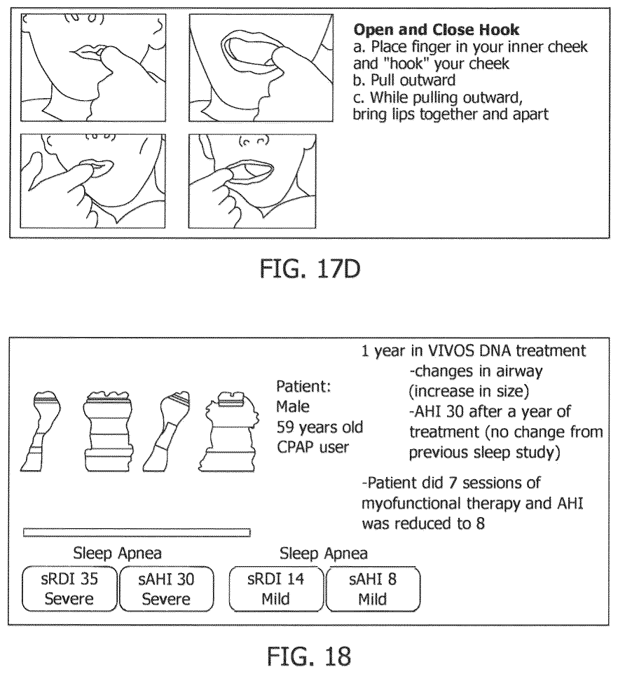

Orofacial myofunctional therapy exercises targeting head posture (illustrated by FIG. 15A) may help improve head posture. Orofacial myofunctional therapy exercises targeting head posture include: head turn with tongue extension (FIG. 15B), neck resistance (FIG. 15C), pectoral stretch (FIG. 15D), and chin tuck (FIG. 15E). Orofacial myofunctional therapy exercises targeting head posture also include: surprise granny (FIG. 17B), upper lip stretch (FIG. 17C), and open and close hook (FIG. 17D).

### Nasal Breathing.

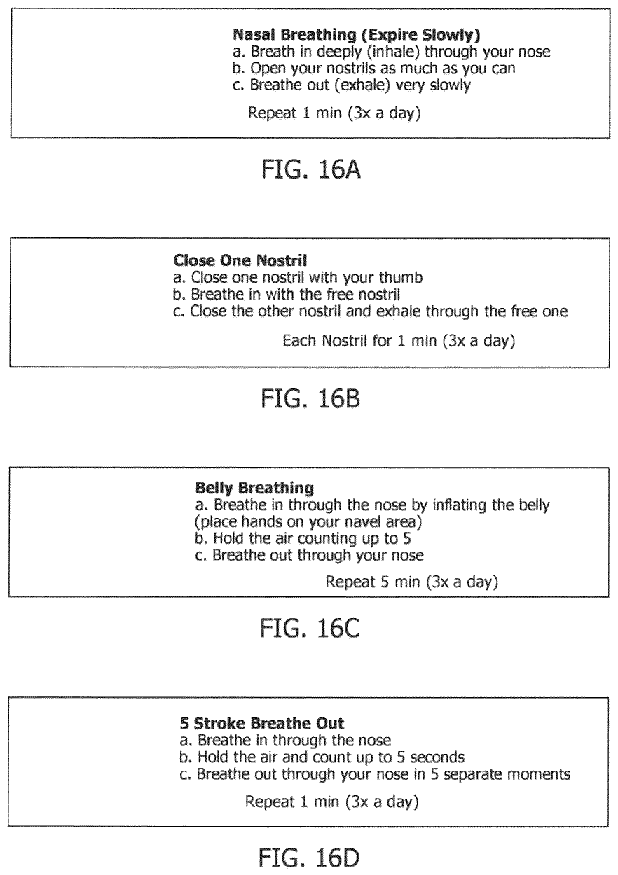

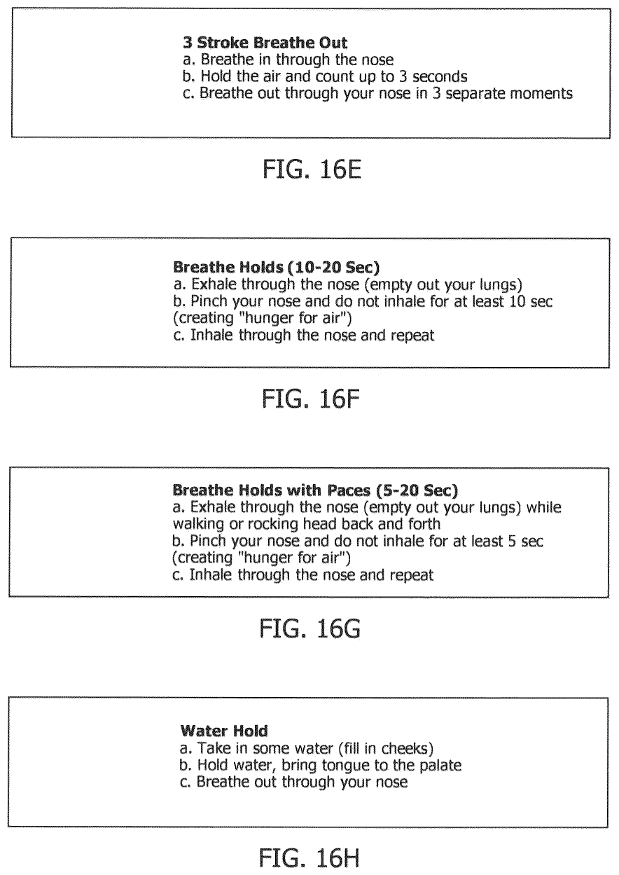

Orofacial myofunctional therapy exercises targeting nasal breathing may help improve nasal breathing. Orofacial myofunctional therapy exercises targeting nasal breathing include: expire slowly (FIG. 16A), close one nostril (FIG. 16B), belly breathing (FIG. 16C), 5 stroke breath out (FIG. 16D), 3 stroke breath out (FIG. 16E), breath holds (10-20 sec) (FIG. 16F), breath holds with paces (5-20 sec) (FIG. 16G), and water hold (FIG. 16H).

### Oral Appliances.

Generally, the oral appliance may be any suitable oral appliance that advances the methods described herein. In some embodiments, the oral appliance is a dental appliance. In some embodiments, the oral appliance is an osteogenetic appliance.

In some embodiments, the oral appliance is selected from the group consisting of oral appliances configured to modify an airway of a patient, oral appliances configured to treat a sleep disordered breathing symptom, oral appliances configured to reposition the jaws, oral appliances configured to improve breathing by opening up the airway, oral appliances configured to treat a temporomandibular disorder, oral appliances configured to reposition an upper and/or lower jaw, oral appliances configured to treat a temporomandibular joint disorder, oral appliances configured to treat a dental or orthodontic condition, aligners, clear aligners, palate expanders, palate shapers, removable oral appliances, daytime-nighttime oral appliances (DNA appliances), vibrational oral appliances, vibrational oral appliances configured to provide mandibular advancement, oral appliances configured to emit infrared light, oral appliances configured to promote bone growth, oral appliances configured for photobiomodulation, and combinations thereof.

In some embodiments, the oral appliance is custom designed to increase the size of, and/or change the shape of, an upper airway of a patient.

Example oral appliances include those disclosed in U.S. Pat. Nos. 7,887,324 and 8,192,196, the contents of which are incorporated herein by reference in their entirety. Other example appliances include a vibrational oral appliance with mandibular advancements.

### Systems.

Generally, the system described herein treats an orofacial muscle disorder in a patient. In some embodiments, the system includes: an oral appliance configured to modify a first component of an oral anatomy of the patient; instructions for performing at least one orofacial myofunctional therapy exercise to exercise a second component of the oral anatomy of the patient; and optionally at least one tool for performing the at least one orofacial myofunctional therapy exercise.

In some embodiments, the instructions are in a form selected from the group consisting of written instructions, electronic instructions, and combinations thereof.

In some embodiments, the system further includes a non-transitory computer-readable storage medium having computer-executable instructions embodied thereon, wherein when executed by a computing device including at least one processor in communication with a memory, the computer-readable instructions cause the computing device to provide the patient with the instructions for performing at least one orofacial myofunctional therapy exercise.

In some embodiments, the system further includes a computing device including: a memory; and a processor communicatively coupled to the memory, the processor programmed to provide the patient with the instructions for performing at least one orofacial myofunctional therapy exercise.

In some embodiments, the computing device is configured to assess at least one health metric of the patient. In some embodiments, the at least one health metric is provided to the processor, which is configured to receive and/or analyze the health metric. In some embodiments, the computing device includes a camera configured to record pictures, videos, and/or three-dimensional images. In some embodiments, the computing device is configured to record pictures and/or videos and/or three-dimensional images of physical features of a patient, such as the teeth, tongue, smile, head, face, oral cavity, posture, range of motion, and combinations thereof. In some embodiments, the computing device is configured to take measurements of physical features including distances, volumes, surface areas, angulations, silhouettes, planar faces, perpendicular dimensions, or combinations thereof. Such measurements may be used for a purpose selected from the group consisting of diagnosis, patient monitoring, patient monitoring throughout therapy, research and development, and combinations thereof.

In some embodiments, the system tracks compliance. The system may track compliance with a compliance tracker included in the oral appliance, a compliance tracker included in the at least one tool, and/or a compliance tracker included in the computing device.

The compliance tracker may be in the form of an embedded chip in the oral appliance and/or the least one tool. The computing device may automatically and/or manually track compliance with the compliance tracker. The computing device may automatically and/or manually track compliance with the compliance tracker to indicate when the oral appliance and/or the least one tool is being utilized. The compliance tracker may take measurements related to the use of the appliance and/or the at least one tool, such as force, pressure, time of use, or combinations thereof.

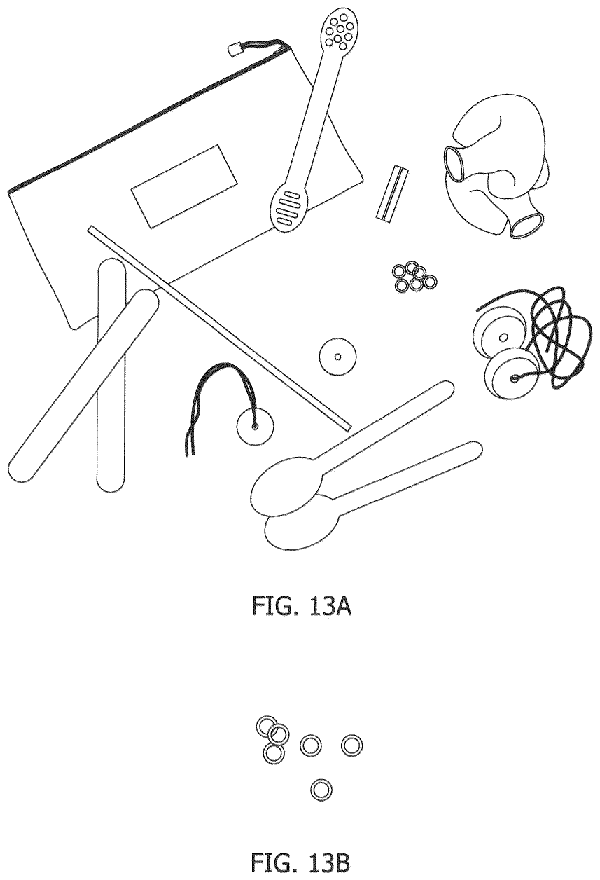

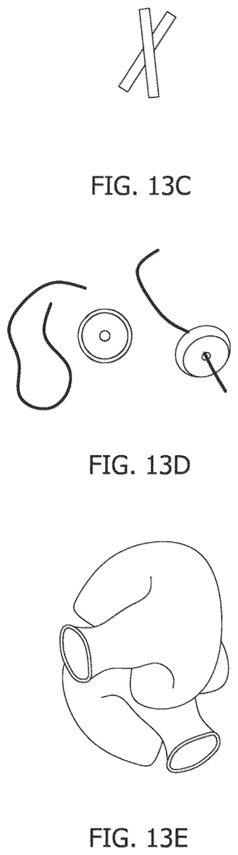

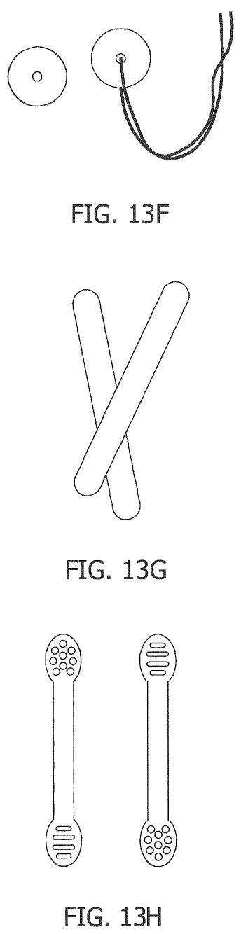

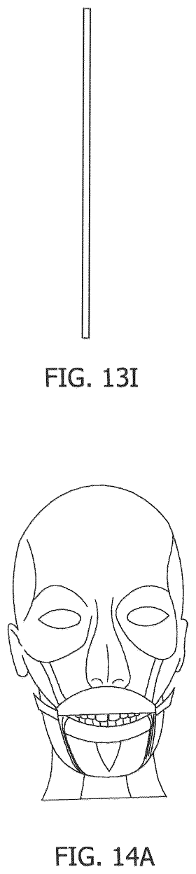

In some embodiments, the orofacial myofunctional therapy exercises may be performed using a tool, such as those depicted in FIG. 13A. Such tools include as rubber bands (FIG. 13B), straws and/or tubes (FIG. 13C), string with weight (FIG. 13D), balloons (FIG. 13E), a button with string (FIG. 13F), tongue depressors (FIG. 13G), duo spoons (FIG. 13H), a rod (FIG. 13I), a Vivos guide (not shown), a bite block (not shown), a wafer (not shown), or combinations thereof.

In some embodiments, the system further includes at least one health metric device. The at least one health metric device is configured to assess at least one health metric of the patient. In some embodiments, the at least one health metric is provided to the processor, which is configured to receive and/or analyze the health metric. In some embodiments, the at least one health metric device includes a breathing or airflow monitor. In some embodiments, the at least one health metric device includes a camera configured to record pictures and/or videos. In some embodiments, the at least one health metric device is configured to record pictures and/or videos of physical features of a patient, such as the teeth, tongue, smile, and range of motion.

In some embodiments, the at least one health metric device is configured to measure airway resistance through a patient's nose or mouth and/or determine lung function and capacity by having the patient respirate or blow into the device. In some embodiments, the at least one health metric device is configured to take a measurement using sonar to measure the volume capacity of the airway. In some embodiments, the measurements of the health metric device are used for a purpose selected from the group consisting of diagnosis, patient monitoring, patient monitoring throughout therapy, research and development, and combinations thereof.

### Applications.

Generally, the non-transitory computer-readable storage medium having computer-executable instructions embodied thereon described herein is configured to treat an orofacial muscle disorder in a patient. In some embodiments, the non-transitory computer-readable storage medium having computer-executable instructions embodied thereon is configured to be used in conjunction with an oral appliance configured to modify a first component of an oral anatomy of the patient and optionally at least one tool for performing at least one orofacial myofunctional therapy exercise. In some embodiments, when executed by a computing device including at least one processor in communication with a memory, the computer-readable instructions cause the computing device to provide the patient with instructions for performing the at least one orofacial myofunctional therapy exercise to exercise a second component of the oral anatomy of the patient.

In some embodiments, the computer-readable instructions further cause the computing device to cause a user interface on a mobile computing device to display feedback regarding the at least one orofacial myofunctional therapy exercise. The mobile computing device and the computing device may be the same device or may be separate devices.

In some embodiments, the computer-readable instructions further cause the computing device to perform at least one operation selected from the group consisting of receive input from the patient, provide a healthcare professional with input from the patient, receive input from the healthcare professional, provide the patient with input from the healthcare professional, facilitate communication between the patient and the healthcare professional, facilitate communication between a guardian of the patient and the healthcare professional, facilitate communication between a guardian of multiple patients and the healthcare professional, facilitate communication and/or collaboration between a first healthcare professional and at least one other healthcare professional, facilitate communication between the patient and a technical support professional, facilitate communication between a guardian of the patient and a technical support professional, facilitate communication between a guardian of multiple patients and a technical support professional, facilitate communication between a healthcare professional and a technical support professional, allow the patient to register for an educational service (e.g. a webinar, a seminar, a therapy session, etc.), allow a healthcare professional to register for an educational service (e.g. a webinar, a seminar, etc.), provide the patient with at least one educational resource, output an animation, output a gamification component, store data and/or information, monitor progress of the patient, and combinations thereof.

When the computer-readable instructions further cause the computing device to facilitate collaboration between a first healthcare professional and a second healthcare professional, the first healthcare professional and/or the second healthcare professional may be different from the healthcare professional.

In some embodiments, the computer-readable instructions further cause the computing device to receive input from the patient. The input from the patient may include patient data, exercise data, patient opinions, patient health history data, patient complaints, patient chief complaints, commerce transactions, e-commerce transactions, therapy feedback, patient consents, patient consent for treatment, patient consent for testimonials, patient consent for release of videos and/or photographs, and combinations thereof.

In some embodiments, the computer-readable instructions further cause the computing device to provide a healthcare professional with input from the patient. The input from the patient may be provided in real-time and/or to a data repository.

In some embodiments, the computer-readable instructions further cause the computing device to receive input from the healthcare professional. The input from the healthcare professional may include healthcare professional data, analyses of exercise data, healthcare professional opinions, patient data, patient health data, x-ray data, intraoral scans, facial 3D scans, intraoral pictures, 2D photos, videos, speech assessments, patient health history data, patient complaints, patient chief complaints, clinical exam information, treatment objectives, treatment plans, treatment progress, and combinations thereof.

In some embodiments, the computer-readable instructions further cause the computing device to provide the patient with input from the healthcare professional. The input from the healthcare professional may be provided in real-time and/or to a data repository.

In some embodiments, the computer-readable instructions further cause the computing device to facilitate communication between the patient and the healthcare professional. The communication between the healthcare professional and the patient may be in real-time and/or via a data repository. The communication may be in a medium selected from the group consisting of text, audio, video, and combinations thereof.

In some embodiments, the computer-readable instructions further cause the computing device to provide the patient with at least one educational resource. The at least one educational resource may be selected from the group consisting of text information, video information, audio information, activity information, exercise information, interactive information, live information, coaching information, therapy information, feedback information, screening information, and combinations thereof.

In some embodiments, the computer-readable instructions further cause the computing device to output an animation. The animation may include an animation selected from the group consisting of animations depicting at least one orofacial myofunctional therapy exercise, animations encouraging patient progress, gamification animations, and combinations thereof.

In some embodiments, the computer-readable instructions further cause the computing device to output a gamification component. In some embodiments, the gamification component is selected from the group consisting of points, rewards, points redeemable for rewards, money, and combinations thereof.

In some embodiments, the gamification component gamifies an activity selected from the group consisting of therapies, exercises, referrals, social media interactions, and combinations thereof.

In some embodiments, the computer-readable instructions further cause the computing device to monitor progress of the patient. Monitoring the progress of the patient may include automatically tracking progress of the patient, automatically tracking progress of the patient via compliance tracking, automatically tracking progress of the patient via at least one sensor in the oral appliance, automatically tracking progress of the patient via at least one sensor in the at least one tool, monitoring the progress of the patient via picture and/or video recording, monitoring the progress of the patient via a health metric device, monitoring the progress of the patient via entries of progress entered by manually by the patient and/or the healthcare professional, or a combination thereof.

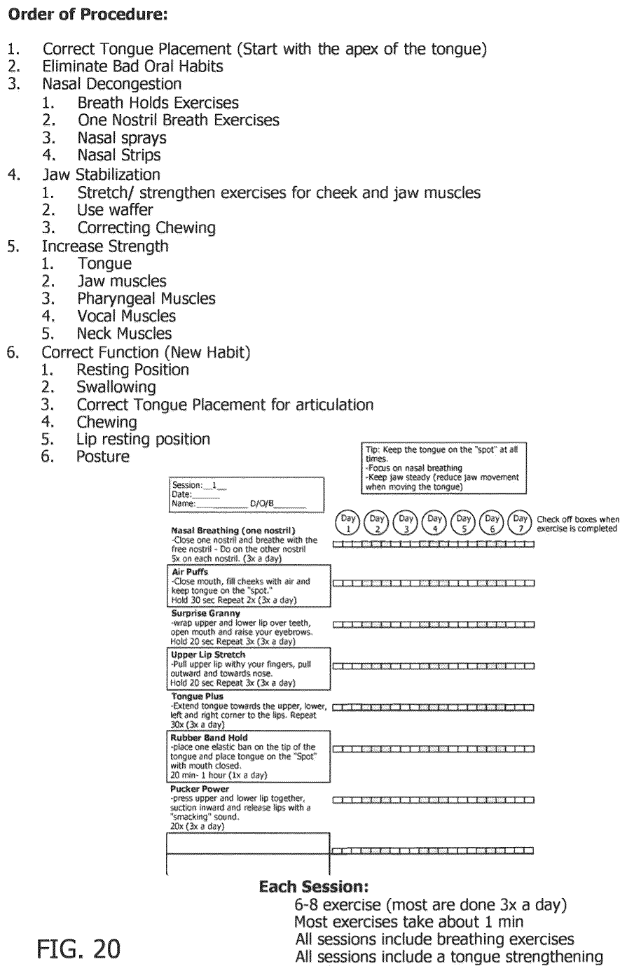

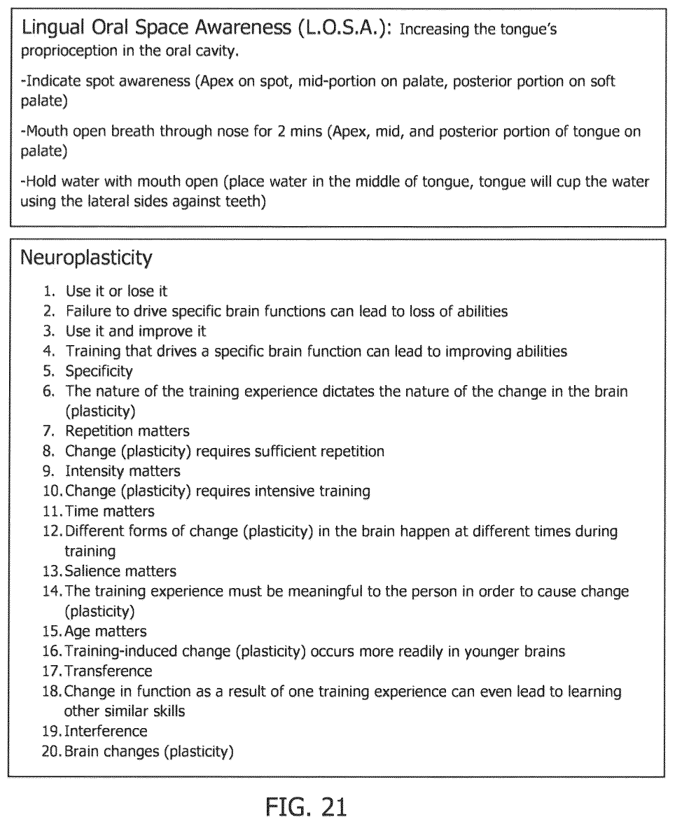

A tracking system and procedure for an orofacial myofunctional therapy program is shown in FIG. 20. The tracking system includes instructions for select orofacial myofunctional therapy exercises, as well as a way to record progress for each exercise.

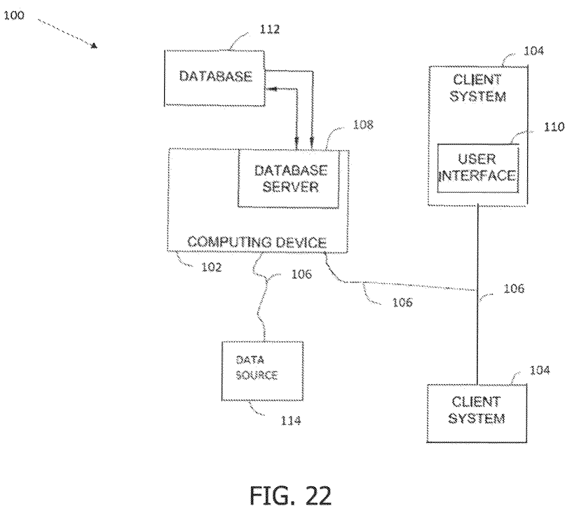

FIG. 22 is a block diagram of an exemplary embodiment of a computer system 100 that includes a computing device 102 in accordance with one exemplary embodiment of the present disclosure. Computer system 100 may be used to implement one or more of the methods described herein.

More specifically, in the exemplary embodiment, system 100 includes computing device 102, and a plurality of client sub-systems, also referred to as client systems 104, connected to computing device 102. In one embodiment, client systems 104 are computers including a web browser, such that computing device 102 is accessible to client systems 104 using the Internet and/or using network 106. Client systems 104 are interconnected to the Internet through many interfaces including a network 106, such as a local area network (LAN) or a wide area network (WAN), dial-in-connections, cable modems, special high-speed Integrated Services Digital Network (ISDN) lines, and RDT networks. Client systems 104 may include external systems used to store data. Computing device 102 is also in communication with one or more data sources 114 using network 106. Further, client systems 104 may additionally communicate with data sources 114 using network 106. Further, in some embodiments, one or more client systems 104 may serve as data sources 114, as described herein. Client systems 104 may be any device capable of interconnecting to the Internet including a web-based phone, PDA, or other web-based connectable equipment.

A database server 108 is connected to a database 112, which contains information on a variety of matters, as described below in greater detail. In one embodiment, centralized database 112 is stored on device 102 and can be accessed by potential users at one of client systems 104 by logging onto computing device 102 through one of client systems 104. In an alternative embodiment, database 112 is stored remotely from device 102 and may be non-centralized. Database 112 may be a database configured to store information used by computing device 102 including, for example, transaction records, as described herein.

Database 112 may include a single database having separated sections or partitions, or may include multiple databases, each being separate from each other. Database 112 may store data received from data sources 114 and generated by computing device 102. For example, database 112 may store patient data, as described in detail herein.

In the exemplary embodiment, client systems 104 may be associated with any party capable of using system 100 as described herein. In the exemplary embodiment, at least one of client systems 104 includes a user interface 110. For example, user interface 110 may include a graphical user interface with interactive functionality, such that input, transmitted from computing device 102 to client system 104, may be shown in a graphical format. A user of client system 104 may interact with user interface 110 to view, explore, and otherwise interact with the displayed information.

In the exemplary embodiment, computing device 102 receives data from a plurality of data sources 114, and aggregates and analyzes the received data as described in detail herein.

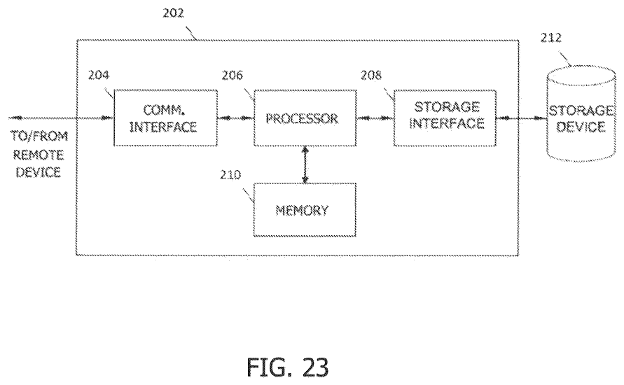

FIG. 23 illustrates an exemplary configuration of a server system 202 such as a computing device, in accordance with one exemplary embodiment of the present disclosure. Server system 202 may be used to implement one or more of the methods described herein. Server system 202 may also include, but is not limited to, a database server (not shown).

Server system 202 includes a processor 206 for executing instructions. Instructions may be stored in a memory area 210, for example. Processor 206 may include one or more processing units (e.g., in a multi-core configuration) for executing instructions. The instructions may be executed within a variety of different operating systems on the server system 202, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

Processor 206 is operatively coupled to a communication interface 204 such that server system 202 is capable of communicating with a remote device such as a user system or another server system 202. For example, communication interface 204 may receive requests from a client system via the Internet (not shown).

Processor 206 may also be operatively coupled to a storage device 212. Storage device 212 is any computer-operated hardware suitable for storing and/or retrieving data. In some embodiments, storage device 212 is integrated in server system 202. For example, server system 202 may include one or more hard disk drives as storage device 212. In other embodiments, storage device 212 is external to server system 202 and may be accessed by a plurality of server systems 202. For example, storage device 212 may include multiple storage units such as hard disks or solid state disks in a redundant array of inexpensive disks (RAID) configuration. Storage device 212 may include a storage area network (SAN) and/or a network attached storage (NAS) system.

In some embodiments, processor 206 is operatively coupled to storage device 212 via a storage interface 208. Storage interface 208 is any component capable of providing processor 206 with access to storage device 212. Storage interface 208 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 206 with access to storage device 212.

Memory area 210 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for storage of a computer program.

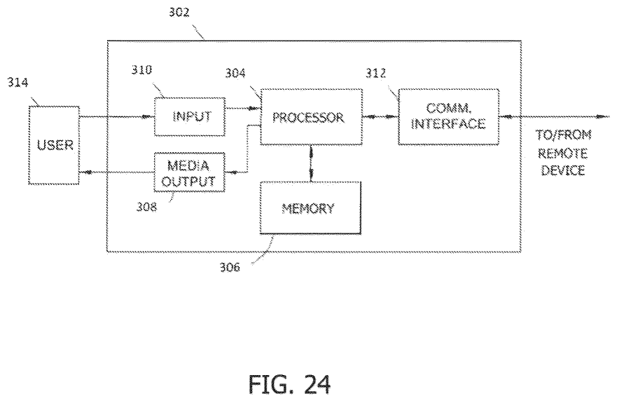

FIG. 24 illustrates an exemplary configuration of a client computing device 302. Client computing device 302 may be used to implement one or more of the methods described herein. Client computing device 302 may include, but is not limited to, client systems (“client computing devices”) 104. Client computing device 302 includes a processor 304 for executing instructions. In some embodiments, executable instructions are stored in a memory area 306. Processor 304 may include one or more processing units (e.g., in a multi-core configuration). Memory area 306 is any device allowing information such as executable instructions and/or other data to be stored and retrieved. Memory area 306 may include one or more computer-readable media.

Client computing device 302 also includes at least one media output component 308 for presenting information to a user 314. Media output component 308 is any component capable of conveying information to user 314. In some embodiments, media output component 308 includes an output adapter such as a video adapter and/or an audio adapter. An output adapter is operatively coupled to processor 304 and operatively couplable to an output device such as a display device (e.g., a liquid crystal display (LCD), organic light emitting diode (OLED) display, cathode ray tube (CRT), or “electronic ink” display) or an audio output device (e.g., a speaker or headphones).

In some embodiments, client computing device 302 includes an input device 310 for receiving input from user 314. Input device 310 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 308 and input device 310.

Client computing device 302 may also include a communication interface 312, which is communicatively couplable to a remote device such as server system 301 or a web server. Communication interface 312 may include, for example, a wired or wireless network adapter or a wireless data transceiver for use with a mobile phone network (e.g., Global System for Mobile communications (GSM), 3G, 4G, 5G, or Bluetooth) or other mobile data network (e.g., Worldwide Interoperability for Microwave Access (WIMAX)).

Stored in memory area 306 are, for example, computer-readable instructions for providing a user interface to user 314 via media output component 308 and, optionally, receiving and processing input from input device 310. A user interface may include, among other possibilities, a web browser and client application. Web browsers enable users 314 to display and interact with media and other information typically embedded on a web page or a website from a web server. A client application allows users 314 to interact with a server application. The user interface, via one or both of a web browser and a client application, facilitates display of information provided by computing device 102. The client application may be capable of operating in both an online mode (in which the client application is in communication with computing device 102) and an offline mode (in which the client application is not in communication with computing device 102).

Further aspects of the present disclosure are provided by the subject matter of the following clauses:

1. A method for treating an orofacial muscle disorder in a patient, the method comprising:
modifying a first component of an oral anatomy of the patient with an oral appliance; and
exercising a second component of the oral anatomy of the patient with at least one orofacial myofunctional therapy exercise.

2. A system for treating an orofacial muscle disorder in a patient, the system comprising:
an oral appliance configured to modify a first component of an oral anatomy of the patient;
instructions for performing at least one orofacial myofunctional therapy exercise to exercise a second component of the oral anatomy of the patient; and
optionally at least one tool for performing the at least one orofacial myofunctional therapy exercise.

3. A non-transitory computer-readable storage medium having computer-executable instructions embodied thereon for treating an orofacial muscle disorder in a patient, wherein the computing device is configured to be used in conjunction with an oral appliance configured to modify a first component of an oral anatomy of the patient and optionally at least one tool for performing at least one orofacial myofunctional therapy exercise, wherein when executed by a computing device including at least one processor in communication with a memory, the computer-readable instructions cause the computing device to:
provide the patient with instructions for performing the at least one orofacial myofunctional therapy exercise to exercise a second component of the oral anatomy of the patient.

4. The subject matter of any preceding clause, wherein the first component of the oral anatomy of the patient is selected from the group consisting of an airway, an airway including a nasal cavity, an oral cavity, and an upper and lower airway, an upper airway, a tooth position, a jaw position, an upper jaw position, a lower jaw position, a tongue position, a lip position, a bite, a component relating to breathing, a component relating to posture, a component relating to speech, a component relating to sleep disordered breathing symptoms, and combinations thereof.

5. The subject matter of any preceding clause, wherein modifying the first component of the oral anatomy of the patient comprises increasing the size of the airway of the patient and/or changing the shape of the airway of the patient.

6. The subject matter of any preceding clause, wherein the first component of the oral anatomy of the patient is modified before, concurrently with, and/or after the second component of the oral anatomy of the patient is exercised.

7. The subject matter of any preceding clause, wherein the oral appliance is selected from the group consisting of oral appliances configured to modify an airway of a patient, oral appliances configured to treat a sleep disordered breathing symptom, oral appliances configured to reposition the jaws, oral appliances configured to improve breathing by opening up the airway, oral appliances configured to treat a temporomandibular disorder, oral appliances configured to reposition an upper and/or lower jaw, oral appliances configured to treat a temporomandibular joint disorder, oral appliances configured to treat a dental or orthodontic condition, aligners, clear aligners, palate expanders, palate shapers, removable oral appliances, daytime-nighttime oral appliances (DNA appliances), vibrational oral appliances, vibrational oral appliances configured to provide mandibular advancement, oral appliances configured to emit infrared light, oral appliances configured to promote bone growth, oral appliances configured for photobiomodulation, and combinations thereof.

8. The subject matter of any preceding clause, wherein exercising the second component of the oral anatomy of the patient comprises using at least one tool to perform the at least one orofacial myofunctional therapy exercise.

9. The subject matter of any preceding clause, wherein exercising the second component of the oral anatomy of the patient comprises exercising the second component of the oral anatomy of the patient with at least one orofacial myofunctional therapy exercise in a multi-phase orofacial myofunctional therapy program.

10. The subject matter of any preceding clause, further comprising providing the patient with the oral appliance.

11. The subject matter of any preceding clause, further comprising providing the patient with instructions to perform the at least one orofacial myofunctional therapy exercise.

12. The subject matter of any preceding clause, wherein the size of the airway of the patient is increased and/or shape of the airway of the patient is changed, and the patient is trained on how to use the increased the size of the airway and/or the changed shape of the airway.

13. The subject matter of any preceding clause, wherein the second component of the oral anatomy of the patient is selected from the group consisting of head posture, resting tongue position, tongue strength, breathing patterns, nasal breathing, lip seal, orofacial muscles, chewing and swallowing patterns, oral noxious habits, functional frenuloplasty, range of motion, tongue range of motion, elasticity in facial muscles, speech, psychological associations thereof, and combinations thereof.

14. The subject matter of any preceding clause, wherein the second component of the oral anatomy of the patient is selected from the group consisting of head muscles, head airways, face muscles, face tissues, nasal muscles, nasal tissues, tongue muscles, tongue tissues, throat muscles, throat tissues, lip muscles, lip tissues, cheek muscles, cheek tissues, jaw muscles, jaw tissues, and combinations thereof.

15. The subject matter of any preceding clause, wherein the second component of the oral anatomy of the patient is selected from the group consisting of orofacial muscles, cranial muscles, neck muscles, a genioglossus muscle, a styloglossus muscle, a hyoglossus muscle, a palatoglossus muscle, a lateral-pterygoid muscle, a masseter muscle, a superficial masseter muscle, a platysma muscle, an orbicularis muscle, a levator labii superioris muscle, a risorius muscle, a zygomaticus major muscle, a zygomaticus minor muscle, a zygomaticus muscle, a depressor anguli oris muscle, a depressor labii inferioris muscle, a mentalist muscle, a superior lingualis muscle, an inferior lingualis muscle, a vertical lingualis muscle, a transverse lingualis muscle, a buccinator muscle, and combinations thereof.

16. The subject matter of any preceding clause, wherein the instructions are in a form selected from the group consisting of written instructions, electronic instructions, and combinations thereof.

17. The subject matter of any preceding clause, further comprising a non-transitory computer-readable storage medium having computer-executable instructions embodied thereon, wherein when executed by a computing device including at least one processor in communication with a memory, the computer-readable instructions cause the computing device to provide the patient with the instructions for performing at least one orofacial myofunctional therapy exercise.

18. The subject matter of any preceding clause, further comprising a computing device comprising:
a memory; and
a processor communicatively coupled to the memory, the processor programmed to provide the patient with the instructions for performing at least one orofacial myofunctional therapy exercise.

19. The subject matter of any preceding clause, wherein the computer-readable instructions further cause the computing device to cause a user interface on a mobile computing device to display feedback regarding the at least one orofacial myofunctional therapy exercise.

20. The subject matter of any preceding clause, wherein the computer-readable instructions further cause the computing device to:
receive input from the patient;
provide a healthcare professional with input from the patient;
receive input from the healthcare professional;
provide the patient with input from the healthcare professional;
facilitate communication between the patient and the healthcare professional;
facilitate communication between a guardian of the patient and the healthcare professional;
facilitate communication between a guardian of multiple patients and the healthcare professional;
facilitate communication and/or collaboration between a first healthcare professional and at least one other healthcare professional;
facilitate communication between the patient and a technical support professional;
facilitate communication between a guardian of the patient and a technical support professional;
facilitate communication between a guardian of multiple patients and a technical support professional;
facilitate communication between a healthcare professional and a technical support professional;
allow the patient to register for an educational service (e.g. a webinar, a seminar, a therapy session, etc.);
allow a healthcare professional to register for an educational service (e.g. a webinar, a seminar, etc.);
provide the patient with at least one educational resource;
output an animation;
output a gamification component;
store data and/or information; and/or
monitor progress of the patient.

21. The subject matter of any preceding clause, wherein the computer-readable instructions further cause the computing device to receive input from the patient.

22. The subject matter of any preceding clause, wherein the computer-readable instructions further cause the computing device to provide a healthcare professional with input from the patient.

23. The subject matter of any preceding clause, wherein the computer-readable instructions further cause the computing device to receive input from the healthcare professional.

24. The subject matter of any preceding clause, wherein the computer-readable instructions further cause the computing device to provide the patient with input from the healthcare professional.

25. The subject matter of any preceding clause, wherein the computer-readable instructions further cause the computing device to facilitate communication between the patient and the healthcare professional.

26. The subject matter of any preceding clause, wherein the computer-readable instructions further cause the computing device to facilitate communication between a guardian of the patient and the healthcare professional.

27. The subject matter of any preceding clause, wherein the computer-readable instructions further cause the computing device to facilitate communication between a guardian of multiple patients and the healthcare professional.

28. The subject matter of any preceding clause, wherein the computer-readable instructions further cause the computing device to facilitate communication and/or collaboration between a first healthcare professional and at least one other healthcare professional.

29. The subject matter of any preceding clause, wherein the computer-readable instructions further cause the computing device to facilitate communication between the patient and a technical support professional.

30. The subject matter of any preceding clause, wherein the computer-readable instructions further cause the computing device to facilitate communication between a guardian of the patient and a technical support professional.

31. The subject matter of any preceding clause, wherein the computer-readable instructions further cause the computing device to facilitate communication between a guardian of multiple patients and a technical support professional.

32. The subject matter of any preceding clause, wherein the computer-readable instructions further cause the computing device to facilitate communication between a healthcare professional and a technical support professional.

33. The subject matter of any preceding clause, wherein the computer-readable instructions further cause the computing device to allow the patient to register for an educational service.

34. The subject matter of any preceding clause, wherein the computer-readable instructions further cause the computing device to allow a healthcare professional to register for an educational service.

35. The subject matter of any preceding clause, wherein the computer-readable instructions further cause the computing device to provide the patient with at least one educational resource.

36. The subject matter of any preceding clause, wherein the computer-readable instructions further cause the computing device to output an animation.

37. The subject matter of any preceding clause, wherein the computer-readable instructions further cause the computing device to output a gamification component.

38. The subject matter of any preceding clause, wherein the computer-readable instructions further cause the computing device to store data and/or information.

39. The subject matter of any preceding clause, wherein the computer-readable instructions further cause the computing device to monitor progress of the patient.

40. The subject matter of any preceding clause, wherein the mobile computing device and the computing device are the same device.

41. The subject matter of any preceding clause, wherein the mobile computing device and the computing device are separate devices.

42. The subject matter of any preceding clause, wherein the at least one educational resource is selected from the group consisting of text information, video information, audio information, activity information, exercise information, interactive information, live information, coaching information, therapy information, feedback information, screening information, and combinations thereof.

43. The subject matter of any preceding clause, wherein compliance is tracked with a compliance tracker included in the oral appliance, a compliance tracker included in the at least one tool, and/or a compliance tracker included in the computing device.

### Examples

Without further elaboration, it is believed that one skilled in the art using the preceding description can utilize the present invention to its fullest extent. The following Examples are, therefore, to be construed as merely illustrative, and not limiting of the disclosure in any way whatsoever. It is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Combination of an Oral Appliance and Orofacial Myofunctional Therapy

Orofacial myofunctional therapy including orofacial myofunctional therapy exercises was combined with an oral appliance. This combination, and the resulting outcomes, are depicted in FIG. 18. The oral appliance was a daytime-night appliance (DNA) configured for changing a patient's airway. The patient was a 59-year-old male CPAP user. The patient wore the DNA for a year of treatment, which resulted in an increase in the size of the patient's airway. Subsequently, the patient performed seven sessions of myofunctional therapy, which resulted in a reduction of the AHI from 30 to 8. Therefore, this combination improved the clinical outcome compared to the use of the oral appliance alone.

### Example 2. Combination of an Oral Appliance and Orofacial Myofunctional Therapy

A comparison between therapy using only an oral appliance and therapy using an oral appliance combined with orofacial myofunctional therapy is shown in Table 1 below. The exemplified oral appliance was a DNA appliance. Sixty-six adult patients were exposed to therapy using only an oral appliance and twenty-one adult patients were exposed to therapy using an oral appliance combined with orofacial myofunctional therapy. Values in AHI were measured according to their obstructive sleep apnea (OSA) classifications: 0-5 is considered normal or resolved, 5-14 is considered mild, 15-29 is considered moderate, and 30 or greater is considered severe.

The results for therapy using only orofacial myofunctional therapy were previously reported by Camacho et al. (“Myofunctional Therapy to Treat Obstructive Sleep Apnea: A Systematic Review and Meta-analysis”, Sleep, Vol. 38, No. 5, 2015). In particular, the meta-analysis described therein found an average AHI decrease of about 50% for one-hundred and twenty adult patients. Similar results were found in children.

The results of Table 1 show that the therapy using an oral appliance combined with orofacial myofunctional therapy resulted in improved patient outcomes compared to therapy using only an oral appliance. In particular, the percent change in AHI was improved for the therapy using an oral appliance combined with orofacial myofunctional therapy compared to the oral appliance alone. The percent change in AHI was about equal for the therapy using an oral appliance combined with orofacial myofunctional therapy compared to the orofacial myofunctional therapy alone. Furthermore, the combined therapy included an improvement in GSA classification.

TABLE 1
Comparisons between therapies.

| | | | Percent | |
|---|---|---|---|---|
| | Percent | | improved by at | |
| | change | | least 1 OSA | Percent |
| | in AHI | p-value | classification | resolved |
| Only Oral Appliance | −34 | 8.4022E−07 | 56 | 20 |
| (DNA) | | | | |
| Only Orofacial | −50 | <0.0001 | Not | Not |
| Myofunctional | | | reported | reported |
| Therapy | | | | |
| Oral Appliance | −47 | 0.0016473 | 86 | 52 |
| (DNA) and Orofacial | | | | |
| Myofunctional | | | | |
| Therapy | | | | |

The methods and systems of the present application provide substantial benefits not previously observed in the art. Orofacial myofunctional therapy combined with the use of an oral appliance has been demonstrated herein to provide improved clinical outcomes over the use of only oral appliances. This combined therapy was found to result in similar reductions in AHI compared to the use of only orofacial myofunctional therapy. Also demonstrated herein is that the combination of an oral appliance and orofacial myofunctional therapy may improve OSA by at least one OSA classification or even resolve OSA. The methods and systems provided herein achieve lowered AHI, respiratory disturbance index (RDI), and oxygen desaturation index (ODI) numbers, which equates to a happier, healthier, more productive, and more focused individual.

This written description uses examples to illustrate the present disclosure, including the best mode, and also to enable any person skilled in the art to practice the disclosure, including making and using any compositions or systems and performing any incorporated methods. The patentable scope of the disclosure is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of excludes any element, step, or ingredient not specified. If in the claim, such would close the claim to the inclusion of materials other than those recited except for impurities ordinarily associated therewith. When the phrase “consisting of appears in a clause of the body of a claim, rather than immediately following the preamble, it limits only the element set forth in that clause; other elements are not excluded from the claim as a whole.

The transitional phrase “consisting essentially of is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

References to “some embodiments” in the above description are not intended to be interpreted as excluding the existence of additional embodiments that also incorporate the recited features.

What is claimed is:

1. A method for treating an orofacial muscle disorder in a patient, the method comprising:
modifying a first component of an oral anatomy of the patient with an oral appliance; and
exercising a second component of the oral anatomy of the patient with at least one orofacial myofunctional therapy exercise.

2. The method of claim 1, wherein the first component of the oral anatomy of the patient is selected from the group consisting of an airway, an airway including a nasal cavity, an oral cavity, and an upper and lower airway, an upper airway, a tooth position, a jaw position, an upper jaw position, a lower jaw position, a tongue position, a lip position, a bite, a component relating to breathing, a component relating to posture, a component relating to speech, a component relating to sleep disordered breathing symptoms, and combinations thereof

3. The method of claim 1, wherein modifying the first component of the oral anatomy of the patient comprises increasing the size of the airway of the patient and/or changing the shape of the airway of the patient.

4. The method of claim 1, wherein the first component of the oral anatomy of the patient is modified before, concurrently with, and/or after the second component of the oral anatomy of the patient is exercised.

5. The method of claim 1, wherein the oral appliance is selected from the group consisting of oral appliances configured to modify an airway of a patient, oral appliances configured to treat a sleep disordered breathing symptom, oral appliances configured to reposition the jaws, oral appliances configured to improve breathing by opening up the airway, oral appliances configured to treat a temporomandibular disorder, oral appliances configured to reposition an upper and/or lower jaw, oral appliances configured to treat a temporomandibular joint disorder, oral appliances configured to treat a dental or orthodontic condition, aligners, clear aligners, palate expanders, palate shapers, removable oral appliances, daytime-nighttime oral appliances (DNA appliances), vibrational oral appliances, vibrational oral appliances configured to provide mandibular advancement, oral appliances configured to emit infrared light, oral appliances configured photobiomodulation, and combinations thereof.

6. The method of claim 1, wherein the second component of the oral anatomy of the patient comprises using at least one tool to perform the at least one orofacial myofunctional therapy exercise.

7. The method of claim 1, wherein exercising the second component of the oral anatomy of the patient comprises exercising the second component of the oral anatomy of the patient with at least one orofacial myofunctional therapy exercise in a multi-phase orofacial myofunctional therapy program.

8. The method of claim 1, further comprising providing the patient with the oral appliance.

9. The method of claim 1, further comprising providing the patient with instructions to perform the at least one orofacial myofunctional therapy exercise.

10. The method of claim 1, wherein the method increases the size of the airway of the patient and/or changes the shape of the airway of the patient, and further trains the patient on how to use the increased the size of the airway and/or the changed shape of the airway.

11. The method of claim 1, wherein the second component of the oral anatomy of the patient is selected from the group consisting of head posture, resting tongue position, tongue strength, breathing patterns, oral noxious habits, functional frenuloplasty, range of motion, tongue range of motion, elasticity in facial muscles, speech psychological associations thereof, and combinations thereof.

12. The method of claim 1, wherein the second component of the oral anatomy of the patient is selected from the group consisting of head muscles, head airways, face muscles, face tissues, nasal muscles, nasal tissues, tongue muscles, tongue tissues, throat muscles, throat tissues, lip muscles, lip tissues, cheek muscles, cheek tissues, jaw muscles, jaw tissues, and combinations thereof.

13. The method of claim 1, wherein the second component of the oral anatomy of the patient is selected from the group consisting of orofacial muscles, cranial muscles, neck muscles, a genioglossus muscle, a styloglossus muscles, a hyoglossus muscle, a palatoglossus muscle, a lateral-pterygoid muscle, a masseter muscle, a superficial masseter muscle, a platysma muscles, an orbicularis muscle, a levator labii superioris muscle, a risorius muscle, a zygomaticus major muscle, a zygomaticus minor muscle, a zygomaticus muscle, a depressor anguli oris muscle, a depressor labii inferioris muscle, a mentalist muscle, a superior lingualis muscle, an inferior lingualis muscle, a vertical lingualis muscle, a transverse lingualis muscle, a buccinator muscle, and combinations thereof.

14. A system for treating an orofacial muscle disorder in a patient, the system comprising:
an oral appliance configured to modify a first component of an oral anatomy of the patient;
instructions for performing at least one orofacial myofunctional therapy exercise to exercise a second component of the oral anatomy of the patient; and
optionally at least one tool for performing the at least one orofacial myofunctional therapy exercise.

15. The system of claim 14, wherein the instructions are in a form selected from the group consisting of written instructions, electronic instructions, and combinations thereof.

16. The system of claim 14, further comprising a non-transitory computer-readable storage medium having computer-executable instructions embodied thereon, wherein when executed by a computing device including at least one processor in communication with a memory, the computer-readable instructions cause the computing device to provide the patient with the instructions for performing at least one orofacial myofunctional therapy exercise.

17. The system of claim 14, further comprising a computing device comprising:
a memory; and
a processor communicatively coupled to the memory, the processor programmed to provide the patient with the instructions for performing at least one orofacial myofunctional therapy exercise.

18. A non-transitory computer-readable storage medium having computer-executable instructions embodied thereon for treating an orofacial muscle disorder in a patient, wherein the computing device is configured to be used in conjunction with an oral appliance configured to modify a first component of an oral anatomy of the patient and optionally at least one tool for performing at least one orofacial myofunctional therapy exercise, wherein when executed by a computing device including at least one processor in communication with a memory, the computer-readable instructions cause the computing device to:
provide the patient with instructions for performing the at least one orofacial myofunctional therapy exercise to exercise a second component of the oral anatomy of the patient.

19. The non-transitory computer-readable storage medium according to claim 18, wherein the computer-readable instructions further cause the computing device to cause a user interface on a mobile computing device to display feedback regarding the at least one orofacial myofunctional therapy exercise.

20. The non-transitory computer-readable storage medium according to claim 18, wherein the computer-readable instructions further cause the computing device to:
receive input from the patient;
provide a healthcare professional with input from the patient;
receive input from the healthcare professional;
provide the patient with input from the healthcare professional;
facilitate communication between the patient and the healthcare professional;
facilitate communication between a guardian of the patient and the healthcare professional;
facilitate communication between a guardian of multiple patients and the healthcare professional;
facilitate communication and/or collaboration between a first healthcare professional and at least one other healthcare professional;
facilitate communication between the patient and a technical support professional;
facilitate communication between a guardian of the patient and a technical support professional;
facilitate communication between a guardian of multiple patients and a technical support professional;
facilitate communication between a healthcare professional and a technical support professional;
allow the patient to register for an educational service;
allow a healthcare professional to register for an educational service;
provide the patient with at least one educational resource;
output an animation;
output a gamification component;
store data and/or information; and/or
monitor progress of the patient.
